(19) United States
(12) Patent Application Publication
(10) Pub. No.: US 2020/0345692 A1
(43) Pub. Date: Nov. 5, 2020

(54) THERAPEUTIC TARGETING OF RECEPTOR TYROSINE KINASE INHIBITOR-INDUCED ANDROGEN RECEPTOR PHOSPHORYATION IN CANCER

(71) Applicant: Indiana University Research and Technology Corporation, Indianapolis, IN (US)

(72) Inventor: Roberto Pili, Indianapolis, IN (US)

(21) Appl. No.: 16/963,075

(22) PCT Filed: Jan. 18, 2019
(86) PCT No.: PCT/US2019/014153

§ 371 Date: Jul. 17, 2020

Related U.S. Application Data
(60) Provisional application No. 62/619,210, filed on Jan. 19, 2018.
(60) Provisional application No. 62/647,282, filed on Mar. 23, 2018.
(60) Provisional application No. 62/679,477, filed on Jun. 1, 2018.

(51) Int. Cl. A61K 31/404; A61K 31/4166; G01N 33/574; A61P 35/00

(52) U.S. Cl. CPC A61K 31/404; A61K 31/4166; A61K 45/06; A61P 35/00; G01N 33/57419

(57) ABSTRACT

Disclosed herein are compositions and methods for treating cancer. More particularly, the present disclosure relates to compositions and methods for treating cancer including prostate cancer, renal cell carcinoma, hepatocellular carcinoma, thyroid cancer, sarcoma breast cancer, and gastrointestinal stromal tumor. The present disclosure also relates to methods for identifying subjects having drug resistant prostate cancer and drug resistant renal cell carcinoma.

### CROSS REFERENCE TO RELATED APPLICATIONS

This application is a U.S. national phase application of PCT/US2019/014153 (published as WO 2019/143908), filed Jan. 18, 2019, which claims priority to U.S. Provisional Application Ser. No. 62/679,477, filed on Jun. 1, 2018, U.S. Provisional Application Ser. No. 62/647,282, filed on Mar. 23, 2018, and U.S. Provisional Application Ser. No. 62/619,210, filed on Jan. 19, 2018, each of which is incorporated by reference in its entirety.

### INCORPORATION OF SEQUENCE LISTING

A paper copy of the Sequence Listing and a computer readable form of the Sequence Listing containing the file named “IURTC_2018-057-02_ST25.txt”, which is 843 bytes in size (as measured in MICROSOFT WINDOWS® EXPLORER), are provided herein and are herein incorporated by reference. This Sequence Listing consists of SEQ ID NO:1-2.

### BACKGROUND

The present disclosure relates generally to cancer. More particularly, the present disclosure relates to compositions and methods for treating cancer including prostate cancer, renal cell carcinoma, hepatocellular carcinoma, thyroid cancer, sarcoma breast cancer, and gastrointestinal stromal tumor. The present disclosure also relates to methods for identifying subjects having drug resistant prostate cancer and drug resistant renal cell carcinoma.

Androgen receptor (AR) plays a crucial role in the development and progression of prostate cancer. AR expression has also been reported in other solid tumors, including renal cell carcinoma (RCC). AR signaling has been reported to promote progression in RCC via the HIF-2α/VEGF signaling pathway, by recruiting vascular endothelial cells, and by altering the AKT/NF-kB signaling axis. However, AR has also been reported to potentially be a good outcome prognosticator in a retrospective analysis of RCC patients, suggesting that the biological role played by AR in RCC remains unclear.

Enzalutamide is a second generation AR antagonist that inhibits AR-ligand interaction and AR transcriptional activity, and has been approved for the treatment of castration-resistant prostate cancer. RTKis, such as sunitinib, represent the main treatment for RCC but inevitably, acquired resistance occurs within the first year of treatment. Several potential mechanisms have been identified to play a role in drug resistance, including upregulation of alternative pathways. Our group has recently reported that epigenetic tumor cell adaptation to RTKis may lead to kinome reprogramming, as well as increased global serine and tyrosine phosphorylation.

### BRIEF DESCRIPTION

The present disclosure relates generally to cancer. More particularly, the present disclosure relates to compositions and methods for treating cancer including prostate cancer, renal cell carcinoma, hepatocellular carcinoma, thyroid cancer, sarcoma breast cancer, and gastrointestinal stromal tumor. The present disclosure also relates to methods for identifying subjects having drug resistant prostate cancer and drug resistant renal cell carcinoma.

In one aspect, the present disclosure is directed to a composition comprising a kinase inhibitor and an androgen receptor antagonist.

In one aspect, the present disclosure is directed to a composition comprising a receptor tyrosine kinase inhibitor and an androgen receptor antagonist.

In one aspect, the present disclosure is directed to a method of treating drug resistant renal cell carcinoma in a subject having or suspected of having drug resistant renal cell carcinoma, the method comprising: administering to the subject a composition comprising an antagonist of an androgen receptor antagonist.

In one aspect, the present disclosure is directed to a method of identifying a subject having or suspected of having drug resistant renal cell carcinoma, the method comprising: obtaining a sample from the subject; and detecting androgen receptor.

In one aspect, the present disclosure is directed to a method of treating prostate cancer in a subject having or suspected of having prostate cancer, the method comprising: administering to the subject a composition comprising an androgen receptor antagonist.

In one aspect, the present disclosure is directed to a method of treating drug resistant prostate cancer in a subject having or suspected of having drug resistant prostate cancer, the method comprising: administering to the subject a composition comprising an androgen receptor antagonist.

In one aspect, the present disclosure is directed to a method of treating a solid tumor in a subject having or suspected of having a solid tumor, the method comprising: administering to the subject a composition comprising an androgen receptor antagonist and a kinase inhibitor.

In one aspect, the present disclosure is directed to a method of identifying a subject having or suspected of having drug resistant prostate cancer, the method comprising: obtaining a sample from the subject; and detecting androgen receptor.

In one aspect, the present disclosure is directed to use of a composition comprising a receptor tyrosine kinase inhibitor and an androgen receptor antagonist to treat cancer.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1I depict association of sunitinib resistance with increased AR expression, and AR inhibition restoration of drug sensitivity in RCC models. RPPA data depicts an increased AR expression in the RP-R-01 RCC PDX model at the time of resistance (FIG. 1A). Immunohistochemistry (FIG. 1B) and qRT-PCR analysis (FIG. 1C) indicated AR expression in RCC PDX models sensitive (ss) and resistant (sr) to sunitinib. mRNA (FIG. 1D), Western blot analysis (FIG. 1E) and proliferation assay (FIG. 1F) on RCC cell lines, depict an increased AR expression in tumor cells that was less sensitive to sunitinib. IC50 for sunitinib correlated with AR status (FIG. 1G). Cell viability assay of RCC cell lines treated with either sunitinib (5 M), enzalutamide (500 nM) or combination, for 48 hrs indicated synergistic decrease in cell viability in the combination group, only in the presence of AR expression. Ki67 immunofluorescence staining and quantitation of 786-0R cells treated with sunitinib, enzalutamide, or combination (FIG. 1H). Proliferation assay with different sunitinib concentrations showed the shift in IC50 between AR expressing 786-0 cell (786-0AR), compared to parental cell line (FIG. 1I). Bar graphs represent the mean±SD. *p<0.05, **p<0.001, ***p<0.005, ****p<0.0001, ns=not significant.

FIGS. 2A-2J depicts increased sunitinib-induced AR expression associated with activation of AR targeted genes and increased AR phosphorylation. Heatmap indicated increased expression of AR target genes in resistant cells compared to the parental 786-0 cell line (FIG. 2A). Top selected genes increased with increased AR (FIG. 2B). q-PCR analysis showed modulation of AR targeted genes (KLK2, KLK4, ZBTB16, MYC) in AR+786-0R and UMRC2 cell lines, following exposure to sunitinib (5 M for 48 hrs) (FIG. 2C) Immunofluorescence staining for AR phospho Ser 81 and AR-C terminal domain in UMRC2 and 786-0R (after 3-4 weeks washout), following exposure to sunitinib (5 M) (48 hrs) (FIG. 2D) Immunofluorescence includes F-actin (green) and Hoechst (blue) staining for cytoplasm and nuclear visualization, respectively. Quantitative analysis by Image J for AR pSer81 (FIG. 2E) and AR (FIG. 2F). Western blot analysis for AR pSer81 and AR-C terminal domain (FIG. 2G). GFP-labelled AR expressed in 786-0 cell: Nuclear protein localization following short exposure (45 minutes) to sunitinib (5 M) (FIG. 2H). Western blot analysis for CDK1 expression in RP-R-R02LM tumors, following sunitinib exposure in vivo (FIG. 2I). Analysis of CDK1 protein expression by Western blot analysis in 786-0 cell line showed an increase in sunitinib-resistant 786-0R in vitro (FIG. 2J). Bar graphs represent the mean±SD. ****p<0.0001.

FIGS. 3A-3I depict sunitinib-induced AR expression was antagonized by enzalutamide via SPOP-mediated proteasome protein degradation. FIG. 3A depicts immunofluorescence quantitative analysis of AR expression in 786-0, 786-0R, and UMRC2 cell lines treated with sunitinib (5 M), enzalutamide (0.5 M), or combination Immunofluorescence staining (FIG. 3B) and quantitative analysis (FIG. 3C) of AR-N terminal domain (red) in 786-0R treated with sunitinib (5 M) and enzalutamide and combination±the proteasome inhibitor MG132 Immunofluorescence includes F-actin (green) and Hoechst (blue) staining for cytoplasm and nuclear visualization, respectively. Immunofluorescence staining (FIG. 3D) and quantitative analysis (FIG. 3E) of AR-N terminal domain in UMRC2 and UMRC2siSPOP, following exposure to sunitinib, enzalutamide, or combination. Proliferation assay in UMRC2 (FIG. 3F) and UMRC2siSPOP cells (FIG. 3G) treated with sunitinib, enzalutamide, or combination. The inhibition of SPOP neutralized the antitumor effect of enzalutamide in the presence of sunitinib. Immunoprecipitation of AR and ubiquitin in 786-0R±sunitinib suggesting increased drug-induced AR ubiquitination (FIG. 3H). Schematic illustration of the proposed mechanism(s) responsible for sunitinib-induced AR activation and enzalutamide-induced AR degradation (FIG. 3I). Bar graphs represent the mean±SD. *p<0.05, **p<0.001, ***p<0.005, ****p<0.0001, ns=not significant.

FIGS. 4A-4F depict enzalutamide restoration of sensitivity to sunitinib in vivo. NSG mice carrying established 786-0 tumors were treated with sunitinib (40 mg/kg by oral gavage; 5 days on/2 days oft), and enzalutamide (10 mg/kg by oral gavage; 2 days on/5 days off, 5 mice/group). Tumor growth curves and end-point tumor weights are depicted in FIG. 4A. FIG. 4B depicts a separate experiment showing treatment of NSG mice carrying established 786-0 tumors with sunitinib (40 mg/kg by oral gavage; 5 days on/2 days oft) until disease progression (≥50% tumor volume from baseline), then mice were randomized to enzalutamide (10 mg/kg by oral gavage; 2 days on/5 days oft) or combination. Tumor growth curves and end-point tumor weights are depicted in FIG. 4B. FIGS. 4C and 4D depict immunofluorescence staining and quantitative analysis for pAR Ser-81 (FIG. 4C) and for TUNEL (apoptosis) (FIG. 4D). FIG. 4E depicts quantitation of inhibition of angiogenesis by CD31 staining (FIG. 4E), sunitinib-resistant tumor cells continued to proliferate in vivo (Ki67 staining; FIG. 4F)). Bar graphs represent the mean±SD. *p<0.05, **p<0.001, ***p<0.005, ****p<0.0001.

FIGS. 5A-5F depict circulating KLK2 as a biomarker for AR expression in RCC. FIG. 5A depicts quantitative analysis of end-point KLK2 serum levels by ELISA in 786-0 tumor bearing animals treated with sunitinib, enzalutamide, or combination (from FIG. 4A). FIG. 5B depicts AR status in RCC cell lines (from FIG. 1) and hk2 expression from tissue culture supernatants assessed by qRT-PCR. FIG. 5C depicts ELISA assessment of circulating KLK2 in vitro from tissue culture supernatant treated with either sunitinib or enzalutamide, indicating increased KLK2 with sunitinib, which was altered with in the presence of enzalutamide. FIG. 5D depicts in vivo data in RCC PDX model of sunitinib acquired resistance (RP-R-02) and intrinsic resistance (RP-R-02LM), indicating increased KLK2 serum concertation with sunitinib treatment. Data is represented as the mean±SEM (n=3). FIG. 5E depicts circulating KLK2 expression in serum of patients who progressed, indicating increased KLK2 compared to non-progressors (FIG. 5F). Bar graphs represent the mean±SD. *p<0.05, **p<0.001, ***p<0.005, ****p<0.0001.

FIG. 6 depicts sunitinib induced phenotypic changes (cell plasticity) in LnCAP (AR+) cells by bright field microscopy and increased refractive index.

FIG. 7 depicts sunitinib induced phenotypic changes (cell plasticity) in RV-1 (ARV7+) cells by bright field microscopy and increased refractive index.

FIG. 8 depicts chronic sunitinib exposure increased AR phosphorylation at Ser-81 residue in full length AR cells LNCaP as shown by immunofluorescence.

FIG. 9 depicts that nuclear localization of full length AR in LNCaP cells upon exposure to DHT was ligand-dependent.

FIG. 10 depicts that exposure to sunitinib induced nuclear localization of AR independently of the presence of the ligand (DHT).

FIG. 11 depicts that AR nuclear localization induced by sunitinib was decreased by concomitant exposure to enzalutamide.

FIG. 12 depicts that DHT induced AR nuclear localization was prevented by enzalutamide.

FIG. 13 depicts the decrease in DHT induced AR nuclear localization by enzalutamide confirmed by Western Blot analysis.

FIG. 14 depicts the increased the activity of enzalutamide in sunitinib-exposed LNCaP cells.

FIG. 15 depicts increased AR phosphorylation at the Ser-81 residue in the RV1 cell line which carries the AR spliced variant V7 upon chronic exposure to sunitinib.

FIG. 16 depicts chronic sunitinib exposure increased global tyrosine phosphorylation in cells bearing AR spliced variant V7, RV-1.

FIG. 17 depicts the impairment of enzalutamide AR nuclear localization by sunitinib.

FIG. 18 depicts that in the presence of sunitinib enzalutamide-resistant RV1 cells reacquire sensitivity to enzalutamide as shown by the decrease in cell proliferation (FIG. 18).

FIG. 19 depicts the measurement of KLK-2 versus absorbance by ELISA.

FIG. 20 depicts an increase in KLK-2 in patients treated with sunitinib indicating AR activation secondary to drug resistance (FIG. 20).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Disclosed are compositions and methods for treating cancer. Compositions and methods can be used to treat cancers such as prostate cancer, renal cell carcinoma, hepatocellular carcinoma, thyroid cancer, sarcoma breast cancer, gastrointestinal stromal tumor, and solid tumors. Also disclosed are methods of identifying a subject having or suspected of having drug resistant prostate cancer drug resistant renal cell carcinoma, the method comprising: obtaining a sample from the subject; and detecting androgen receptor.

As used herein, “a subject in need thereof” refers to a subject having, susceptible to or at risk of a specified disease, disorder, or condition. More particularly, in the present disclosure the methods of screening biomarkers is to be used with a subset of subjects who have, are susceptible to or are at an elevated risk for experiencing cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors. Such subjects can be susceptible to or at elevated risk for cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), and combinations thereof.

As used herein, “a reference expression level of a biomarker” refers to the expression level of a biomarker established for a subject without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors, expression level of a biomarker in a normal/healthy subject without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors as determined by one skilled in the art using established methods as described herein, and/or a known expression level of a biomarker obtained from literature. The reference expression level of the biomarker can also refer to the expression level of the biomarker established for any combination of subjects such as a subject without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors, expression level of the biomarker in a normal/healthy subject without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors, and expression level of the biomarker for a subject without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors at the time the sample is obtained from the subject, but who later exhibits without cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors. The reference expression level of the biomarker can also refer to the expression level of the biomarker obtained from the subject to which the method is applied. As such, the change within a subject from visit to visit can indicate an increased or decreased risk for cancers such as prostate cancer including drug resistant prostate cancer, renal cell carcinoma including drug resistant renal cell carcinoma, hepatocellular carcinoma, thyroid cancer, sarcoma breast cancer, gastrointestinal stromal tumor, and solid tumors. For example, a plurality of expression levels of a biomarker can be obtained from a plurality of samples obtained from the same subject and used to identify differences between the pluralities of expression levels in each sample. Thus, in some embodiments, two or more samples obtained from the same subject can provide an expression level(s) of a blood biomarker and a reference expression level(s) of the blood biomarker.

In one aspect, the present disclosure is directed to a composition comprising a kinase inhibitor and an androgen receptor antagonist.

In one aspect, the kinase inhibitor is a receptor tyrosine kinase inhibitor.

Suitable receptor tyrosine kinase inhibitors include sunitinib (N-(2-Diethylaminoethyl)-5-[(Z)-(5-fluoro-2-oxo-1H-indol-3-ylidene)methyl]-2,4-dimethyl-1H-pyrrole-3-carboxamide), axitinib (N-Methyl-2-[[3-[(E)-2-pyridin-2-ylethenyl]-1H-indazol-6-yl]sulfanyl]benzamide), pazopanib (5-({4-[(2,3-Dimethyl-2H-indazol-6-yl)methylamino]pyrimidin-2-yl}amino)-2-methylbenzenesulfonamide), cabozantinib (N-(4-((6,7-Dimethoxyquinolin-4-yl)oxy)phenyl)-N′-(4-fluorophenyl)cyclopropane-1,1-dicarboxamide), lenvatinib (4-[3-Chloro-4-(cyclopropylcarbamoylamino)phenoxy]-7-methoxy-quinoline-6-carboxamide), sorafenib (4-[4-[[4-chloro-3-(trifluoromethyl)phenyl]carbamoylamino]phenoxy]-N-methyl-pyridine-2-carboxamide), regorafenib (4-[4-({[4-Chloro-3-(trifluoromethyl)phenyl]carbamoyl}amino)-3-fluorophenoxy]-N-methylpyridine-2-carboxamide hydrate), imatinib (4-[(4-methylpiperazin-1-yl)methyl]-N-(4-methyl-3-{[4-(pyridin-3-yl)pyrimidin-2-yl]amino}phenyl)benzamide), dasatinib (N-(2-chloro-6-methylphenyl)-2-[[6-[4-(2-hydroxyethyl)-1-piperazinyl]-2-methyl-4-pyrimidinyl]amino]-5-thiazole carboxamide monohydrate), nilotinib (4-methyl-N-[3-(4-methyl-1H-imidazol-1-yl)-5-(trifluoromethyl)phenyl]-3-[(4-pyridin-3-ylpyrimidin-2-yl) amino]benzamide), bosutinib (4-[(2,4-dichloro-5-methoxyphenyl)amino]-6-methoxy-7-[3-(4-methylpiperazin-1-yl)propoxy]quinoline-3-carbonitrile), ponatinib (3-(2-Imidazo[1,2-b]pyridazin-3-ylethynyl)-4-methyl-N-[4-[(4-methylpiperazin-1-yl)methyl]-3-(trifluoromethyl)phenyl]benzamide), ruxolitinib ((3R)-3-Cyclopentyl-3-[4-(7H-pyrrolo[2,3-d]pyrimidin-4-yl)pyrazol-1-yl]propanenitrile), tofacitinib (3-[(3R,4R)-4-Methyl-3-[methyl(7H-pyrrolo[2,3-d]pyrimidin-4-yl)amino]piperidin-1-yl]-3-oxopropanenitrile), gefitinib (N-(3-chloro-4-fluoro-phenyl)-7-methoxy-6-(3-morpholin-4-ylpropoxy)quinazolin-4-amine), erlotinib (N-(3-ethynylphenyl)-6,7-bis(2-methoxyethoxy)quinazolin-4-amine), lapatinib (N-[3-Chloro-4-[(3-fluorophenyl)methoxy]phenyl]-6-[5-[(2-methylsulfonylethylamino)methyl]-2-furyl]quinazolin-4-amine), vandetanib (N-(4-bromo-2-fluorophenyl)-6-methoxy-7-[(1-methylpiperidin-4-yl)methoxy]quinazolin-4-amine), afatinib (N-[4-[(3-Chloro-4-fluorophenyl)amino]-7-[[(3S)-tetrahydro-3-furanyl]oxy]-6-quinazolinyl]-4(dimethylamino)-2-butenamide), nintedanib (Methyl (3Z)-3-{1[(4-{methyl[(4-methylpiperazin-1-yl)acetyl]amino}phenyl)amino](phenyl)methylidene}-2-oxo-2,3-dihydro-1H-indole-6-carboxylate), crizotinib (3-[(1R)-1-(2,6-dichloro-3-fluorophenyl)ethoxy]-5-(1-piperidin-4-ylpyrazol-4-yl)pyridin-2-amine), ceritinib (5-chloro-N²-{5-methyl-4-(piperidin-4-yl)-2-[(propan-2-yl)oxy]phenyl}-N⁴-[2-(propane-2-sulfonyl)phenyl]pyrimidine-2,4-diamine), and ibrutinib (1-[(3R)-3-[4-Amino-3-(4-phenoxyphenyl)-1H-pyrazolo[3,4-d]pyrimidin-1-yl]piperidin-1-yl]prop-2-en-1-one). Suitable in vitro dosages range from about 0.5 micromolar to about 10 micromolar. Suitable dosages of sunitinib include 37.5 mg PO QD (oral administration every day) 4 weeks ON, 2 weeks OFF to 50 mg PO QD 4 weeks ON, 2 weeks OFF. Suitable dosages of axitinib include 5 mg PO QD to 10 mg PO QD. Suitable dosage of pazopanib includes 800 Mg PO QD. Suitable dosage of cabozantinib includes about 60 mg PO QD. Suitable dosage of lenvatinib includes 18 mg PO QD. Suitable dosage of sorafenib includes from about 200 mg to about 800 mg PO OD. Suitable dosage of regorafenib includes about 80 mg to about 160 mg PO QD. Suitable dosage of imatinib includes about 100 mg to about 800 mg PO QD. Suitable dosage of dasatinib includes about 20 mg to about 180 mg PO QD. Suitable dosage of nilotinib includes about 100 mg to about 800 mg PO QD. Suitable dosage of bosutinib includes about 300 mg to about 600 mg PO OD. Suitable dosage of ponatinib includes about 15 mg to about 45 mg PO OD. Suitable dosage of ruxolitinib includes about 10 mg to about 50 mg PO OD. Suitable dosage of tofacitinib includes about 10 mg to about 20 mg PO OD. Suitable dosage of gefitinib includes about 250 mg to about 500 mg PO OD. Suitable dosage of erlotinib includes about 100 mg to about 150 mg PO OD. Suitable dosage of lapatinib includes about 500 mg to about 1500 mg PO OD. Suitable dosage of vandetanib includes about 100 mg to about 300 mg PO OD. Suitable dosage of afatinib includes about 10 mg to about 40 mg PO OD. Suitable dosage of nintedanib includes about 100 mg to about 300 mg PO OD. Suitable dosage of crizotinib includes about 200 mg to about 500 mg PO OD. Suitable dosage of ceritinib includes about 150 mg to about 750 mg PO OD. Suitable dosage of ibrutinib includes about 70 mg to about 560 mg PO OD.

In one embodiment, the kinase inhibitor is a serine/threonine kinase inhibitor. Suitable serine/threonine kinase inhibitors include vemurafenib (N-(3-{[5-(4-Chlorophenyl)-1H-pyrrolo[2,3-b]pyridin-3-yl]carbonyl}-2,4-difluorophenyl)propane-1-sulfonamide), trametinib (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide), sirolimus ((1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.0-4,9]hexatriaconta-16,24,26,28-tetraene-2,3,10,14,20-pentone), temsirolimus ((1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), everolimus (Dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone), and palbociclib (6-Acetyl-8-cyclopentyl-5-methyl-2-{[5-(1-piperazinyl)-2-pyridinyl]amino}pyrido[2,3-d]pyrimidin-7(8H)-one). Suitable vemurafenib dose includes about 480 mg to about 960 mg PO g12hr. Suitable trametinib dose includes about 1 mg to about 2 mg PO OD. Suitable sirolimus dose includes about 3 mg/m² loading dose to about 15 mg PO loading dose. Suitable temsirolimus dose includes about 5 mg/week to about 25 mg/week IV. Suitable everolimus dose includes about 0.75 mg PO g12hr to about 10 mg PO GqDay. Suitable palbociclib dose includes about 75 mg PO GqDay to about 125 mg PO GqDay.

In one embodiment, the kinase inhibitor is a lipid kinase inhibitor. Suitable lipid kinase inhibitors include idelalisib (5-Fluoro-3-phenyl-2-[(1S)-1-(7H-purin-6-ylamino)propyl]-4(3H)-quinazolinone) and buparlisib (5-[2,6-bis(morpholin-4-yl)pyrimidin-4-yl]-4-(trifluoromethyl)pyridin-2-amine). Suitable idelalisib dose includes about 100 mg to about 150 mg PO BID. Suitable buparlisib dose includes about 50 mg to about 800 mg PO BID.

In one embodiment, the kinase inhibitor is a combination of a receptor tyrosine kinase inhibitor, a serine/threonine kinase inhibitor, and a lipid kinase inhibitor. Suitable receptor tyrosine kinase inhibitors, a serine/threonine kinase inhibitors, and a lipid kinase inhibitors and doses are described herein.

Suitable androgen receptor antagonists include enzalutamide (4-(3-(4-cyano-3-(trifluoromethyl)phenyl)-5,5-dimethyl-4-oxo-2-thioxoimidazolidin-1-yl)-2-fluoro-N-methylbenzamide), bicalutamide ((RS)—N-[4-cyano-3-(trifluoromethyl)phenyl]-3-[(4-fluorophenyl)sulfonyl]-2-hydroxy-2-methylpropanamide), apalutamide (4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide), abiraterone (abiraterone acetate; [(3S,8R,9S,10R,13S,14S)-10,13-dimethyl-17-pyridin-3-yl-2,3,4,7,8,9,11,12,14,15-decahydro-1H-cyclopenta[a]phenanthren-3-yl] acetate) and ODM-201 (darolutamide; N—((S)-1-(3-(3-Chloro-4-cyanophenyl)-1H-pyrazol-1-yl)propan-2-yl)-5-(1-hydroxyethyl)-1H-pyrazole-3-carboxamide). Suitable dosage of enzalutamide ranges from about 80 mg PO QD to about 1000 mg PO QD. Suitable dosage of bicalutamide includes about 50 mg PO QD. Suitable dosage of apalutamide includes about 240 mg PO QD. Suitable dosage of abiraterone acetate is from about 1000 mg/day. Suitable dosage of ODM-201 ranges from about 200 mg/day to about 1800 mg/day.

In one aspect, the present disclosure is directed to a method of treating renal cell carcinoma in a subject having or suspected of having drug resistant renal cell carcinoma. The method includes: administering to the subject a composition comprising an androgen receptor antagonist.

In one embodiment, the subject has or is suspected of having drug resistant renal cell carcinoma.

Suitable androgen receptor antagonists and doses are described herein.

In one embodiment, the subject is receiving or has received a receptor tyrosine kinase inhibitor therapy. Suitable receptor tyrosine kinase inhibitor therapies and doses are described herein.

In one embodiment, the administration is co-administration of the androgen receptor antagonist and the receptor tyrosine kinase inhibitor therapy. In one embodiment, the co-administration is simultaneous administration of the androgen receptor antagonist and the receptor tyrosine kinase inhibitor therapy. In one embodiment, the co-administration is sequential administration of the androgen receptor antagonist and the receptor tyrosine kinase inhibitor therapy.

The method can further include detecting androgen receptor phosphorylation in a sample obtained from the subject. In one embodiment, phosphorylation of serine 81 of the androgen receptor is detected. Serine 81 numbering of androgen receptor is described in Chen et al. (2012, J. Biol. Chem. 287(11):8571-8583), which describes at least one suitable method for detecting phosphorylation of serine 81 of the androgen receptor.

The method can further include reducing androgen receptor ubiquitin ligase level. In one embodiment, the androgen receptor ubiquitin ligase is Speckle-Type POZ (SPOP). Androgen receptor ubiquitin ligase level can be reduced by gene knockdown methods such as by siRNA.

The method can further include detecting expression of cyclin-dependent kinase 1 (CDK1), kallikrein 2 (KLK2), kallikrein 4 (KLK4), Zinc Finger And BTB Domain Containing 16 (ZBTB16), MYC Proto-Oncogene, BHLH Transcription Factor (MYC), and combinations thereof in a sample obtained from the subject. In one embodiment, the CDK1, KLK2, KLK4, ZBTB16, and MYC expression is increased in the subject having or suspected of having drug resistant renal cell carcinoma. In one embodiment, the CDK1, KLK2, KLK4, ZBTB16, and MYC expression is decreased in the subject having or suspected of having drug resistant renal cell carcinoma following administration of the androgen receptor antagonist.

Suitable samples include whole blood, serum, and plasma.

The method can further include administering dalteparin to the subject. Dalteparin (e.g., dalteparin sodium) is a low molecular weight heparin (i.e., heparin salts having an average molecular weight of less than 8000 Da and for which at least 60% of all chains have a molecular weight less than 8000 Da). Suitable dosages of dalteparin include about 120 international units/kg of body weight subcutaneously every 12 hours to about 10,000 international units/kg of body weight subcutaneously every 12 hours (75 mg once a day to 165 mg once a day)

In one aspect, the present disclosure is directed to a method of identifying a subject having or suspected of having drug resistant renal cell carcinoma. The method includes: obtaining a sample from the subject; detecting androgen receptor expression, detecting androgen receptor phosphorylation, and combinations thereof.

In one embodiment, the subject is receiving a receptor tyrosine kinase inhibitor therapy. In one embodiment, the subject has received a receptor tyrosine kinase inhibitor therapy. The receptor tyrosine kinase inhibitor therapies are described herein.

In one embodiment, the subject is identified as having drug resistant renal cell carcinoma if the androgen receptor expression is increased. In one embodiment, the subject is identified as having drug resistant renal cell carcinoma if the androgen receptor is phosphorylated at serine 81. Serine 81 numbering of androgen receptor is described in Chen et al. (2012, J. Biol. Chem. 287(11):8571-8583), which describes at least one suitable method for detecting phosphorylation of serine 81 of the androgen receptor.

Suitable samples include whole blood, serum, and plasma.

The method can further include detecting expression of cyclin-dependent kinase 1 (CDK1), kallikrein 2 (KLK2), kallikrein 4 (KLK4), Zinc Finger And BTB Domain Containing 16 (ZBTB16), MYC Proto-Oncogene, BHLH Transcription Factor (MYC), and combinations thereof in a sample obtained from the subject. In one embodiment, the CDK1, KLK2, KLK4, ZBTB16, and MYC expression is increased in the subject having or suspected of having drug resistant renal cell carcinoma.

In one aspect, the present disclosure is directed to a method of treating drug resistant prostate cancer in a subject having or suspected of having drug resistant prostate cancer. The method includes: administering to the subject a composition comprising an androgen receptor antagonist.

In one embodiment, the subject has or is suspected of having drug resistant prostate cancer.

Suitable androgen receptor antagonists and doses as described herein.

In one embodiment, the subject is receiving or has received a receptor tyrosine kinase inhibitor therapy, wherein the receptor tyrosine kinase inhibitor therapy is any one of sunitinib, axitinib, pazopanib, cabozantinib, lenvatinib, sorafenib, regorafenib, imatinib, dasatinib, nilotinib, bosutinib, ponatinib, ruxolitinib, tofacitinib, gefitinib, erlotinib, lapatinib, vandetanib, afatinib, nintedanib, crizotinib, ceritinib, and ibrutinib.

In one embodiment, the administration is co-administration of the androgen receptor antagonist and the receptor tyrosine kinase inhibitor therapy. In one embodiment, the co-administration is simultaneous administration of the androgen receptor antagonist and the receptor tyrosine kinase inhibitor therapy. In one embodiment, the co-administration is sequential administration of the androgen receptor antagonist and the receptor tyrosine kinase inhibitor therapy.

The method can further include detecting androgen receptor phosphorylation in a sample obtained from the subject. In one embodiment, phosphorylation of serine 81 of the androgen receptor is detected. Serine 81 numbering of androgen receptor is described in Chen et al. (2012, J. Biol. Chem. 287(11):8571-8583), which describes at least one suitable method for detecting phosphorylation of serine 81 of the androgen receptor.

Suitable samples include a prostate biopsy, whole blood, serum, and plasma.

In one aspect, the present disclosure is directed to a method of identifying a subject having or suspected of having drug resistant prostate cancer. The method includes: obtaining a sample from the subject; detecting androgen receptor expression, detecting androgen receptor phosphorylation, and combinations thereof.

In one embodiment, the androgen receptor detected is the AR-V7 splice variant.

In one embodiment, the subject is receiving a receptor tyrosine kinase inhibitor therapy. In one embodiment, the subject has received a receptor tyrosine kinase inhibitor therapy. The receptor tyrosine kinase inhibitor therapy includes receiving any of sunitinib, axitinib, pazopanib, cabozantinib, lenvatinib, sorafenib, regorafenib, imatinib, dasatinib, nilotinib, bosutinib, ponatinib, ruxolitinib, tofacitinib, gefitinib, erlotinib, lapatinib, vandetanib, afatinib, nintedanib, crizotinib, ceritinib, and ibrutinib.

Suitable samples include a prostate biopsy, whole blood, serum, and plasma.

The method can further include analyzing a sample obtained from the subject for kallikrein 2 expression. In one embodiment, the sample is obtained prior to treatment. In one embodiment, the sample is obtained following treatment. In one embodiment, a first sample is obtained prior to treatment and at least a second sample is obtained following treatment.

In one aspect, the present disclosure is directed to a method of treating a subject having or suspected of having a solid tumor. The method includes: administering to the subject a composition comprising an androgen receptor antagonist and a kinase inhibitor.

In one embodiment, the subject has or is suspected of having hepatocellular carcinoma, thyroid cancer, sarcoma, breast cancer, or gastrointestinal stromal tumor (GIST).

Suitable androgen receptor antagonists and doses are described herein.

In one embodiment, the kinase inhibitor is a receptor tyrosine kinase inhibitor. Suitable receptor tyrosine kinase inhibitors and doses are described herein.

In one embodiment, the kinase inhibitor is a serine/threonine kinase inhibitor. Suitable serine/threonine kinase inhibitors and doses are described herein.

In one embodiment, the kinase inhibitor is a lipid kinase inhibitor. Suitable lipid kinase inhibitors and doses are described herein.

In one embodiment, the kinase inhibitor is a combination of a receptor tyrosine kinase inhibitor, a serine/threonine kinase inhibitor, and a lipid kinase inhibitor.

The method can further include analyzing a sample obtained from the subject for kallikrein 2 expression. In one embodiment, the sample is obtained prior to treatment. In one embodiment, the sample is obtained following treatment. In one embodiment, a first sample is obtained prior to treatment and at least a second sample is obtained following treatment.

Suitable samples include blood, plasma, and serum.

### EXAMPLES

### Materials and Methods

In vitro assays were performed using commercially available RCC cell lines 786-0, UMRC2, ACHN, Caki2 (ATCC) and generated 786-0R (acquired sunitinib resistant). Cells were maintained and cultured in the appropriate media, supplemented with 10% FBS and 1% penicillin and streptomycin. All cells are routinely tested and checked for the absence of mycoplasma. For transient transfection, cells were transfected with 50 nM siSPOP (Ambion) or siControl (Ambion) using Lipofectamine 2000 transfection reagent, according to manufacturer's protocol (cat #:11668027, ThermoFisher Scientific). For treatment, cells were plated in 24 well plated and 24 hours post seeding cells were treated with either sunitinib (5 μM, LC laboratories), enzalutamide (500 nM, Selleckchem), axitinib (5 M, LC laboratories) or MG-132 (10 M, Selleckchem) or combinations, for either 24, 48 or 72 hours. Crystal violet assay (Sigma) was used to evaluate cells growth after different time point-treatment, and absorbance was read using a spectrometer (xMarks Spectrometer, Bio-Rad). For in vivo studies, 786-0 (sunitinib sensitive) and RP-R-02LM (sunitinib resistant) models were used. All in vivo experiments were approved and performed in strict accordance with the guidelines of the Institutional Animal Care and Use Committee (IACUC) at Indiana University, Indianapolis Ind. Six-week-old homozygous ICR, Severe Combined Immune-deficient (SCID) female mice were housed in a sterile, pathogen-free facility and maintained in a temperature controlled room, under a 12-hour light/dark schedule with water and food ad libitum. 786-0 and RP-R-02LM viable tumors were selected, dissected into ˜1 mm² tumor pieces, and implanted subcutaneously into mice. All mice were operated under sedation with oxygen, isoflurane and buprenorphine. When tumors were established, and reached 50 mm², mice were randomly grouped and placed in either control group or treatment groups (n=5-10). Mice received sunitinib treatment (40 mg/kg 5 days on, 2 days off), enzalutamide (MDV300) treatment (10 mg/kg), or a combination of sunitinib and enzalutamide. Tumor burden was assessed once a week by caliper measurement of two diameters of the tumor (L×W=mm²) and reported as tumor volume ((L×W2)/2=mm³). Body weights were assessed using a weighing scale and recorded in grams. Endpoint tumor weights were assessed using a weigh scale and recorded in grams. Tissue and blood were collected under aseptic conditions. 1 ml of blood was collected by cardiac bleeds (terminal) at the end of the experiment. Serum and plasma were separated, and aliquots were stored at −80° C. for further analysis. Tumor tissues were excised, weighed, and cut into sections. Sections were snap-frozen and stored in −80° C., fixed in 10% buffered formalin, or zinc for histopathology and saved in trizol for RNA analysis.

Steroid standards, dihydrotestosterone, progesterone, testosterone and epi-testosterone were purchased from Steraloids (Newport, R.I.). Steroid 13C3-internal-standards were purchased from IsoSciences (King of Prussia, Pa.). Hydroxylamine hydrochloride, ultrapure methanol and water (Chromasolv) were purchased from Sigma-Aldrich (St Louis, Mo.). Steroids were extracted from sample homogenates after addition of internal-standard (0.5 ng each) using tert-butyl methyl ether, and the separated organic layer was evaporated. The extracts were subsequently derivatized using hydroxylamine hydrochloride in water/methanol (26). An Agilent 6495 triple quadrupole mass spectrometer (Santa Clara, Calif.), equipped with a Jet Stream electrospray ion source, a 1290 Infinity II ultrahigh-performance liquid chromatography system, and MassHunter Workstation software was used to quantify steroids. Chromatographic separation of testosterone, epi-testosterone, dihydrotestosterone and progesterone oximes result in elution at 3.4, 3.6, 3.9 and 4.3 minutes, respectively. Molecular ion transitions monitored for progesterone (m/z 345.2 to 124.2), DHT (m/z 306.2 to 255.2), testosterone (m/z 304.2 to 124.1), epi-testosterone (304.2 to 124.1). The same ion transitions plus 3 mass units were monitored for 13C3 internal-standards. The lower limit of quantification was 2.0 femtograms for testosterone, epi-testosterone and progesterone, and 25 femtograms for dihydrotestosterone.

Tissue specimens were fixed for 24 hours, paraffin embedded and sectioned (4 μm). Sections were de-paraffinized and rehydrated through graded alcohol washes. Antigen unmasking was achieved by boiling slides in either sodium citrate buffer (pH=6.0) or EDTA. For immunohistochemistry staining (IHC), sections were further incubated in hydrogen peroxide to reduce endogenous activity. To examine the expressions of our proteins of interests, tissue section was blocked with 2.5% horse serum (Vector Laboratories) and incubated overnight in primary antibodies against AR (1:1000; cat #5153Cell Signaling). Following primary incubation, tissue sections were incubated in horseradish-conjugated anti-rabbit, according to manufacturer's protocol (Vector Laboratories), followed by enzymatic development in diaminobenzidine (DAB) and counter stained in hematoxylin. Sections were dehydrated and mounted with cytoseal 60 (ThermoScientific). For immunofluorescence staining (IF), sections were blocked with 5% BSA (Sigma), stained with either phospho-Tyrosine (1:50; sc-508, Santa Cruz), phospho-Serine (1:50, 600-401-26, Rockland, USA), or AR (1:400; 5153, Cell Signaling), AR-C19 (1:10; sc-815, Santa Cruz), Ki67 (1:10; MA5-14520, ThermoFisher), Tunel (cat # G3250, Promega), Phospho-AR (pS81) (1:50, 04-078, Millipore), and incubated overnight at 40 C. Following primary incubation, sections were incubated with either Alexa Fluor or FITC fluorophores conjugated anti-rabbit (ThermoFisher) or anti-mouse (1:400; ThermoFisher) antibody, at room temperature in a humid light-tight box. Afterwards, slides were stained with actin green (1:10; cat # R37110, ThermoFisher), counter stained with Hoechst (cat #23491-45.4; SIGMA), and mounted with VectorShield mounting medium (Vector laboratories). Stained sections were analyzed either under bright field (IHC), or under appropriate fluorescence wavelength (IF) using the EVOS FL cell imaging microscope (Life Technology) and Leica Confocal microscope (Leica). The number of positive cells was determined in a blinded fashion, by analyzing four random 20× fields per tissue and quantified using Image J software.

RNA was extracted in accordance with manufacturer's protocol (miRNeasy; Qiagen), and RNA sequencing was performed as previously described (25). In brief, RNA illumine sequencing reads were de-multiplex, aligned against human genome (hg19), and results aligned to BAM formatted sequence alignment map via cufflinks program. Differential expressed transcripts were identified between 786-0 and 786-0R samples, and ranked based on the square root of the sum of squares for the log 2 fold change. For qRT-PCR analysis, gene expression assessment on AR target genes was performed using the Prime PCR array (Bio-Rad) according to manufacturer's protocol. AR primer used is forward primer; 5-GGTGAGCAGAGTGCCCTATC-3 (SEQ ID NO:1) and reverse primer; 5-TCGGGTATTTCGCATGTCCC-3 (SEQ ID NO:2). In brief, the denaturation step was carried out at 95° C. for 10 seconds; the annealing step was carried out at 58° C. for 30 seconds, and extension step at 72° C. for 1 minute using the applied Biosystems 7900HT fast real-time PCR system (Applied Biosystems). Sequence Detection Systems Software v2.3 was used to identify cycle threshold (Ct) values and generate gene expression curves. All data were normalized to either GAPDH expression.

Whole cell protein extracts from tissue and cell were denatured, separated on SDS-PAGE gels and transferred to nitrocellulose membranes. After blocking in 5% enhanced blocking agent (GE) in Tris-buffered saline-Tween, membranes were probed overnight at 4° C. with either, AR (1;1000; cat #5153, Cell Signaling), SPOP (1:1000; ab168619, Abcam), or phospho-AR (S81) (1:1000; cell signaling, CA USA). After incubation with the appropriate secondary antibody, results were detected using Western Lightning Chemiluminescence Reagent Plus, according to the manufacturer's instructions (ThermoFisher Scientific) and captured on film. Quantitative measurements of Western blot analysis were performed using ImageJ and Graph-Pad software (Prism 7).

Data analyses are expressed as the mean+standard error of mean (SEM). Statistical significance where appropriate was evaluated using a two-tailed student t test when comparing two groups, or by one-way analysis of variance (ANOVA), using the student-Newman Keuls post-test for multiple comparison. A p value <0.05, *p<0.05, **p<0.01, ***p <0.001, was considered significant; ns=not significant. Statistical analyses were done by GraphPad software.

### Example 1

In this Example, markers associated with drug resistance in renal cell carcinoma were identified.

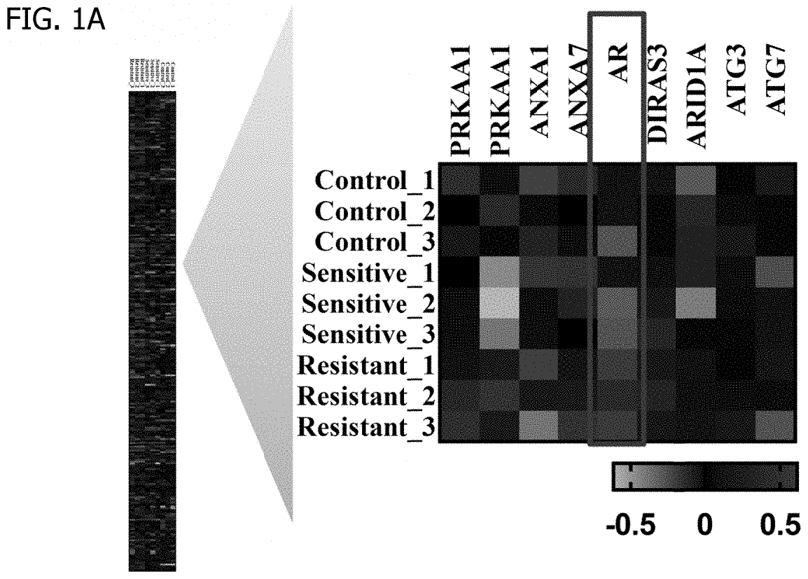

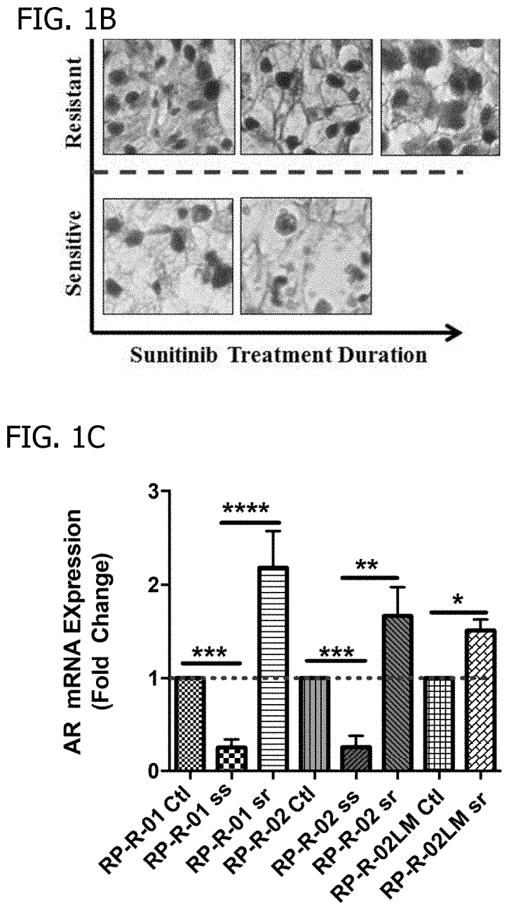

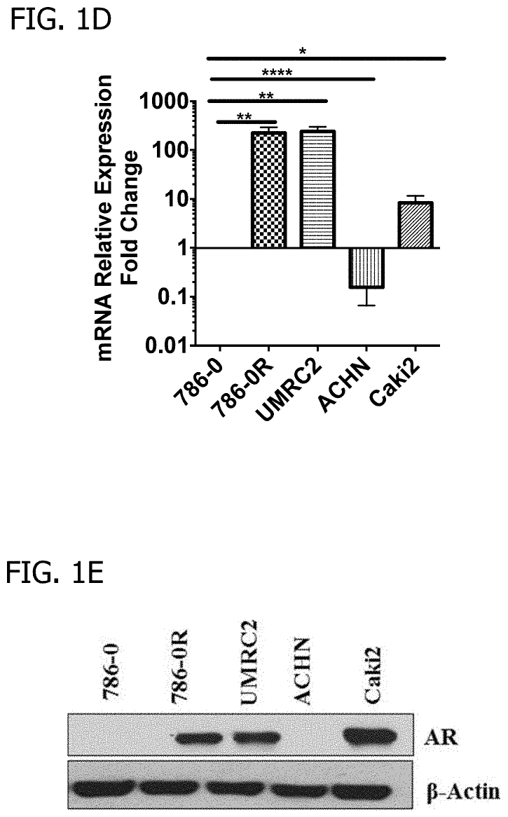

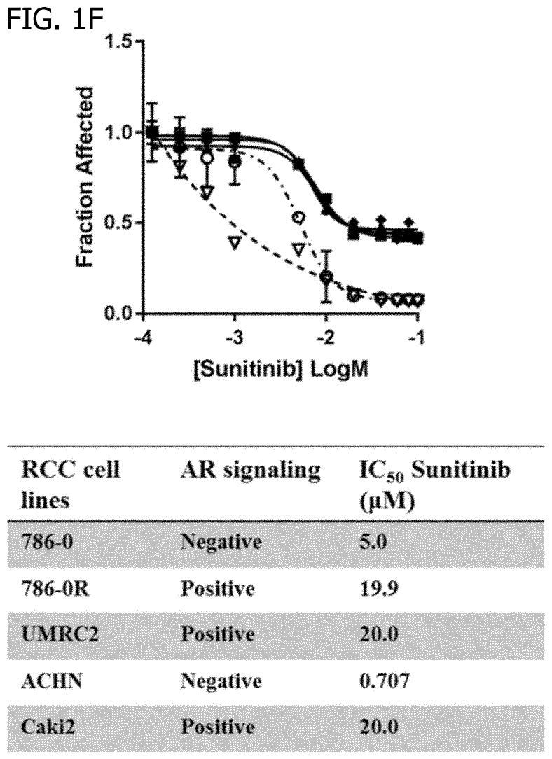

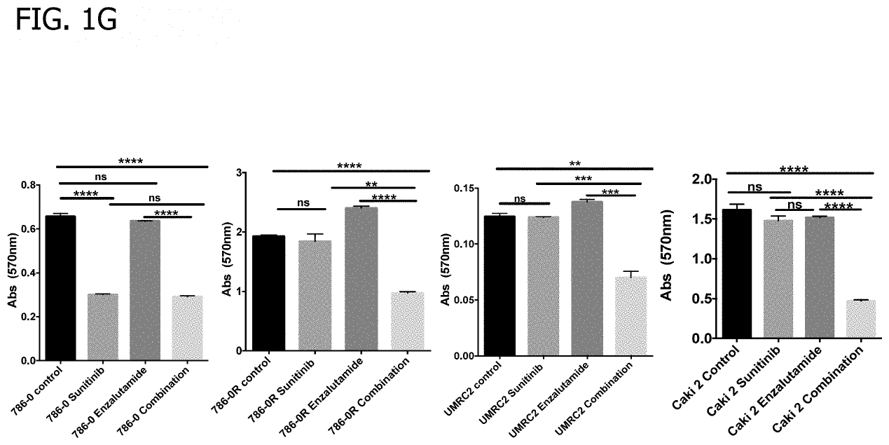

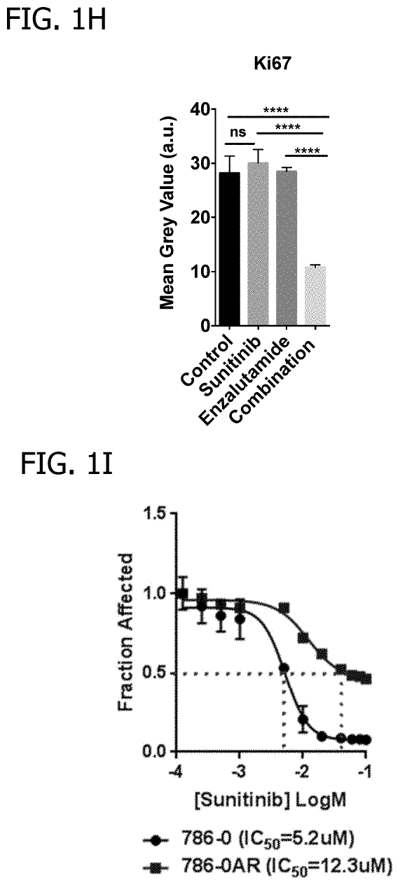

To identify potential markers associated with drug resistance in RCC, an RPPA analysis was performed in a patient-derived xenograft (PDX) model (RP-R-01), where in vivo transient acquired resistance to sunitinib was observed following chronic drug exposure. Dynamic changes were detected in several proteins as tumors progressed from RTKi sensitivity to acquired resistance. Unexpectedly, among the protein changes, there was a significant increase (p>0.05) in AR expression in the sunitinib resistant tumors (FIG. 1A). This was confirmed by immunohistochemistry and qPCR in both RP-R-01 and RP-R-02 tumor models at the time of resistance to sunitinib and in a derived metastatic model (RP-R-02LM) that was intrinsically resistant to sunitinib (FIGS. 1B and 1C). AR gene and protein AR expression were analyzed in the human RCC cell line 786-0 and the sunitinib resistant derivative (786-0R). A significant increase following chronic drug exposure was detected (FIGS. 1D and 1E). AR expression was also assessed in other RCC cell lines (FIG. 1E). AR levels were associated with sensitivity to sunitinib, showing higher IC50 in AR+ RCC cell lines (786-0R, UMRC2 and Caki2) as compared to AR-RCC cell lines (786-0, ACHN) (FIG. 1F). These data indicated that higher AR expression was associated with resistance to the direct anti-tumor effect of sunitinib. To determine whether AR has biological activity in the RCC models, AR activity was inhibited using the AR antagonist, enzalutamide. AR-786-0 and ACHN, AR+786-0R, UMRC2, and Caki2 cells were treated with sunitinib alone, enzalutamide alone, or a combination of sunitinib and enzalutamide for 48 hours, and analyzed using a crystal violet assay. Quantitative analysis indicated a synergistic effect of enzalutamide and sunitinib in sunitinib resistant (AR+) RCC cell lines, but not in sunitinib sensitive (AR-) RCC cell lines (FIG. 1G). Enzalutamide alone did not have a significant effect on AR+786-0R RCC cell viability, but the combination of enzalutamide and sunitinib inhibited Ki67 expression (FIG. 1H). Similar results were obtained with another AR antagonist, bicalutamide, and also with axitinib, another RTKi approved for the treatment of RCC. To further explore the contribution of AR in modulating resistance to sunitinib, ARwt was overexpressed in 786-0 cells (AR-) using a pEGFP-C-AR expressing plasmid. Following 3 weeks of selection, qRT-PCR was performed to confirm successful transfection. A sunitinib dose-response assay was conducted to determine whether AR overexpression decreased sensitivity to sunitinib. Dose response curves indicated a shift in the IC50 from 5.2 M in the 786-0 (parental) to 12.3 M in 786-0AR (FIG. 1I). Taken together, these data indicated that AR expression modified, in part, sunitinib resistance in RCC.

### Example 2

In this Example, functional activity of AR expression in the RCC models was determined.

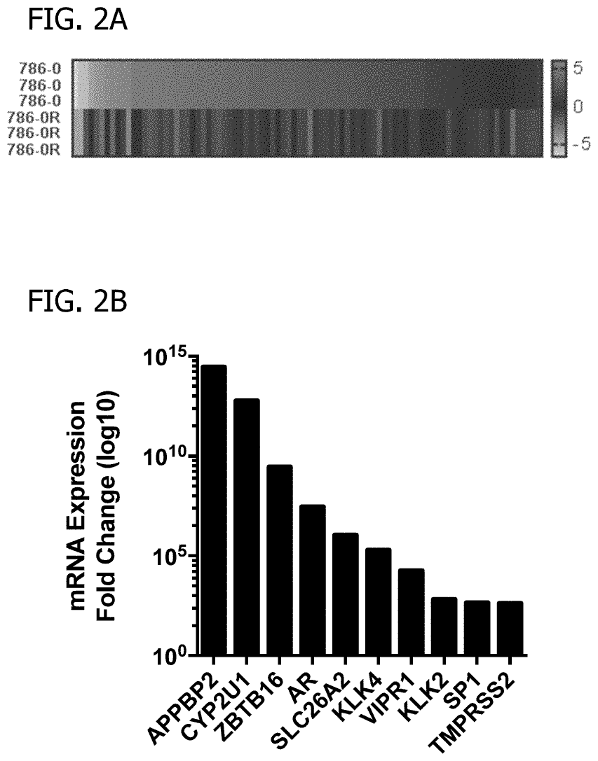

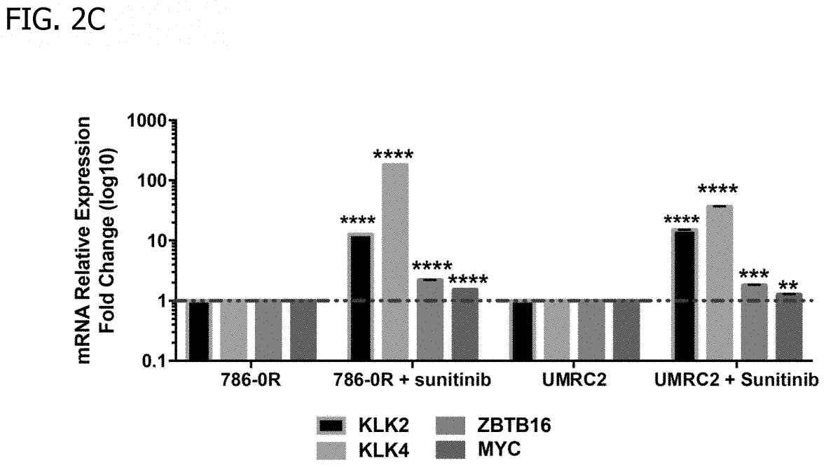

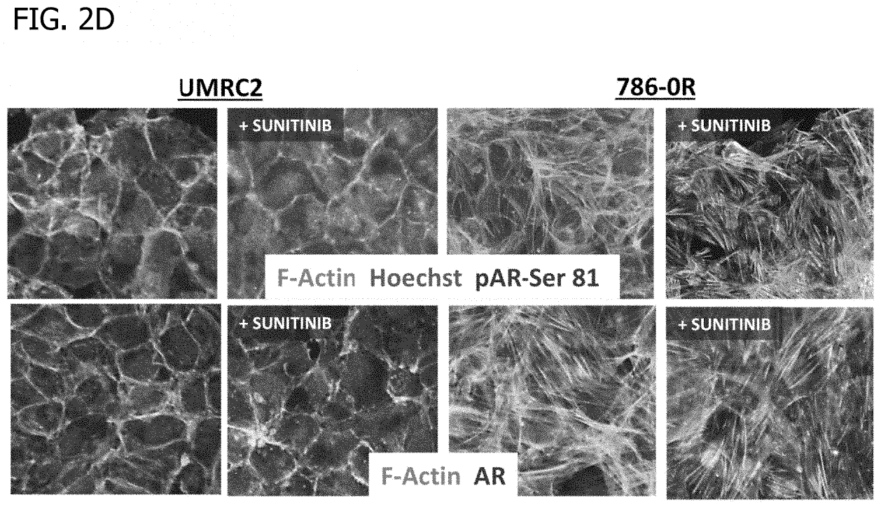

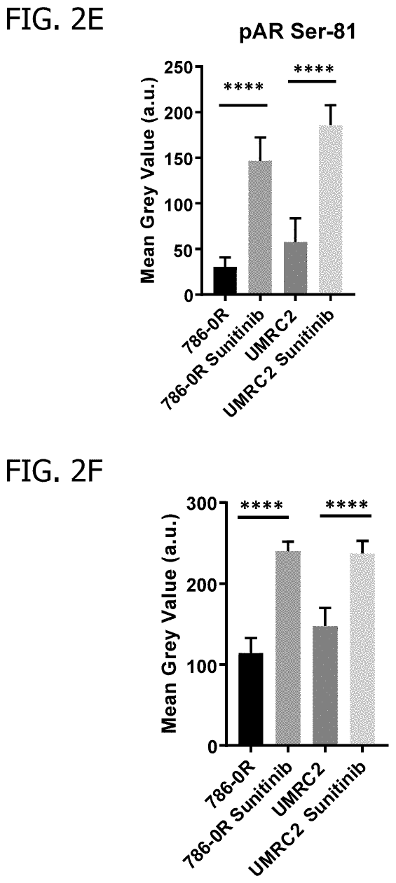

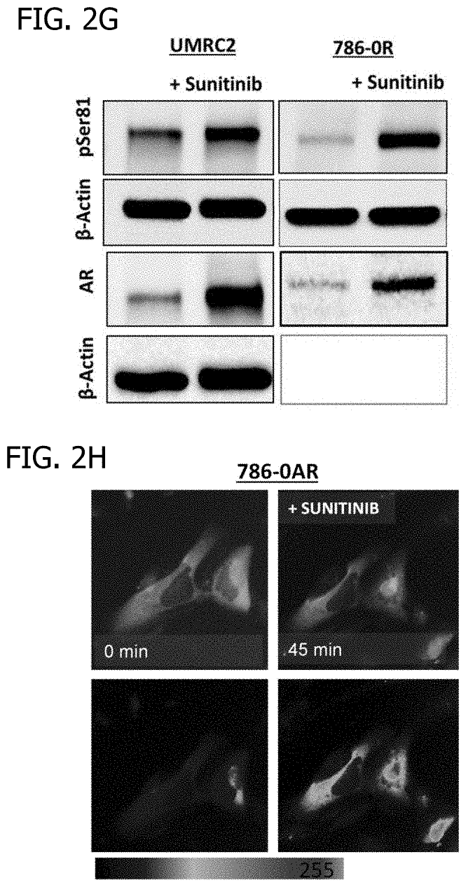

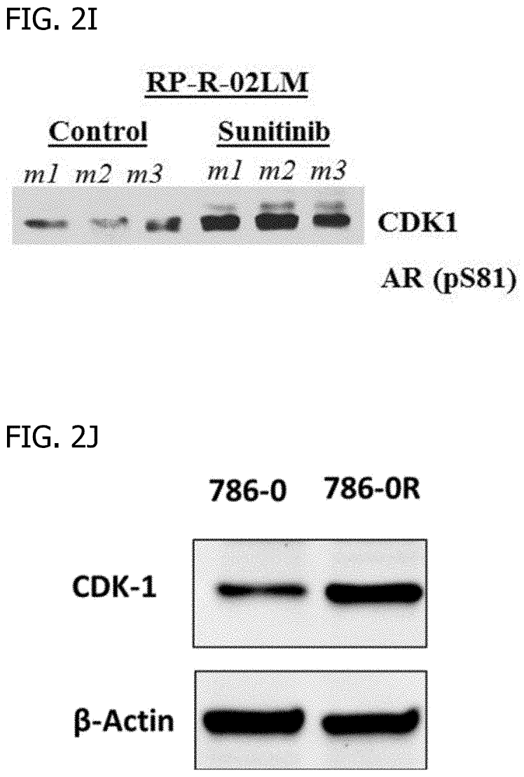

RNA-seq data was analyzed and a gene array analysis was performed on AR signaling and AR targeted genes, comparing the AR sunitinib sensitive 786-0 cell line and the derived AR+ sunitinib-resistant 786-0R cell line. The generated heat map indicated an increase in mRNA expression levels of AR targeted genes (i.e. APPBP2, ZBTB16, KLK4, KLK2, TMPRSS2), indicating that sunitinib-induced AR was transcriptionally active (FIGS. 2A and 2B). To determine whether sunitinib has a direct effect on AR expression, 786-0, 786-0R (after sunitinib washout for one week), and UMRC2 cells were treated with sunitinib. Gene expression of AR targeted genes was then examined in the presence or absence of sunitinib treatment. Quantitative gene expression data showed increased gene expression of AR-driven KLK2, KLK4, ZBTB16 and MYC (2-100 fold) in both 786-0R and UMRC2 cells (FIG. 2C).

AR activation in prostate cancer is generally driven by dihydrotestosterone binding, nuclear translocation, and dimerization leading to DNA binding. To determine whether AR activation required the presence of androgens, the RCC cell lines were cultured in charcoal stripped media. Unexpectedly, the absence of androgens did not influence the cell growth of either AR- or AR+ cell lines, and neither modulated AR gene expression. To further determine the contribution of androgens to AR activity in RCC, mass-spectrometry analysis was performed on PDX (RP-R-01, RP-R-02 with acquired sunitinib resistance, and RP-R-02LM with intrinsic sunitinib resistance), and tumor cell lines (786-0, 786-0R, UMRC2 and UMRC2R). No significant presence of testosterone, epitestosterone or progesterone was detected. These data indicated that AR activity in RCC following sunitinib was likely due to ligand-independent mechanisms.

To address the potential mechanism(s) of sunitinib induced AR activation, phosphorylation of AR following sunitinib resistance was assessed. Immunofluorescence analysis revealed a significant increase in both total nuclear AR expression and phosphorylation of AR at serine residue 81 (pS81 AR), with sunitinib treatment in AR+ UMRC2 and 786-0R (after sunitinib washed out) cell lines (FIG. 2D-2F). Increased total and pS81 AR was confirmed by Western blot analysis (FIG. 2G). AR phosphorylation has been implicated in nuclear translocation in prostate cancer. In 786-0, following a transient GFP-labeled AR transfection, a short treatment with sunitinib indicated a strong nuclear localization of AR (FIG. 2H). To determine whether the increase in CDK1 was associated with sunitinib resistance and induced AR phosphorylation, the RP-RP-02LM tumors treated in vivo with sunitinib were analyzed. In this intrinsically sunitinib-resistant tumor, drug treatment led to increased CDK1 protein expression (FIG. 2I). A similar increase of CDK1 in sunitinib-resistant 786-0R cells was observed as compared to 786-0 cells in vitro (FIG. 2J). These data indicated that sunitinib induces ligand-independent AR activation, and consequent nuclear translocation, likely via S81 phosphorylation.

### Example 3

In this Example, the effect of enzalutamide on AR activity/expression was determined.

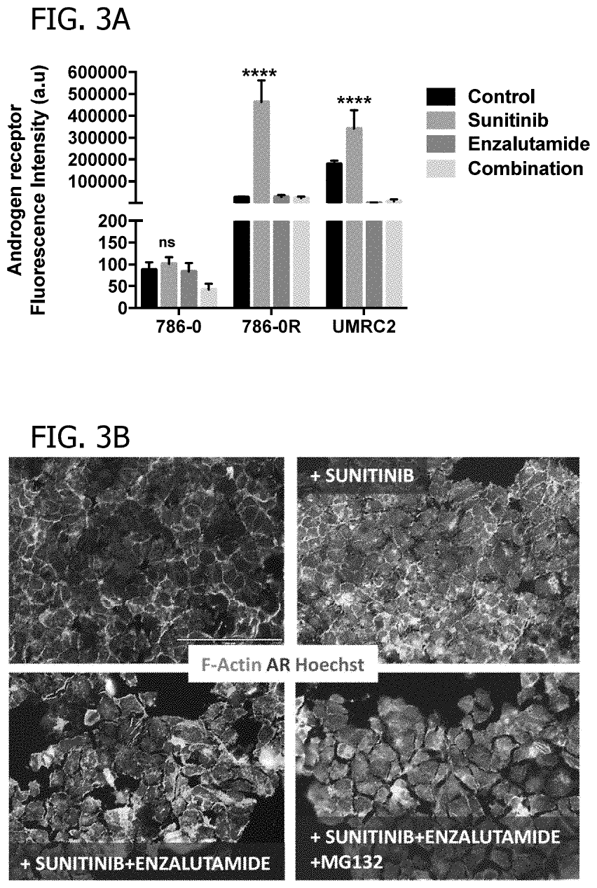

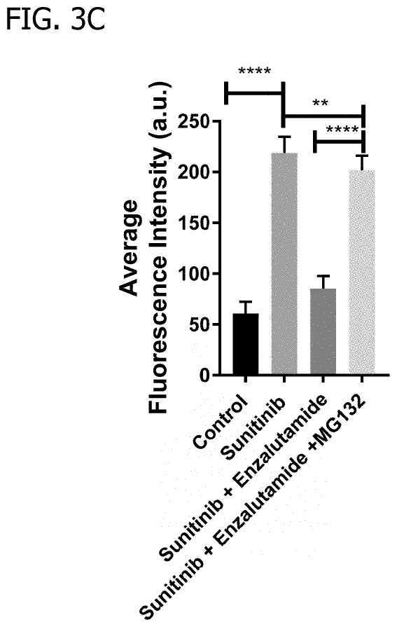

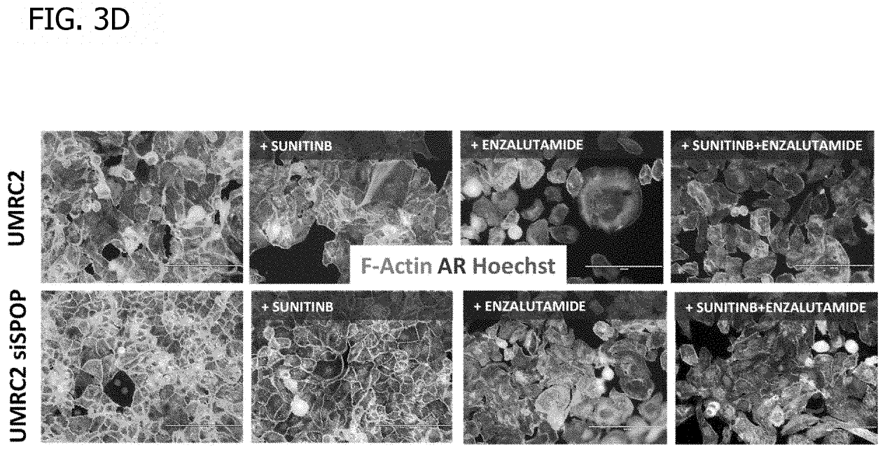

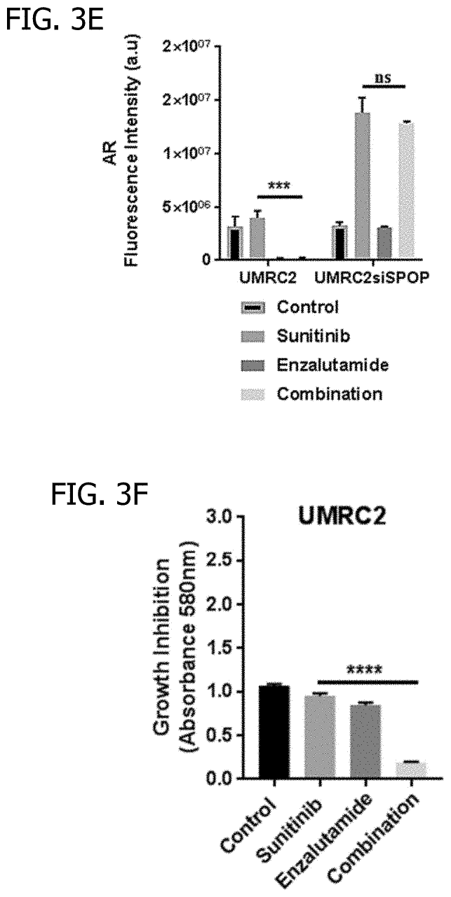

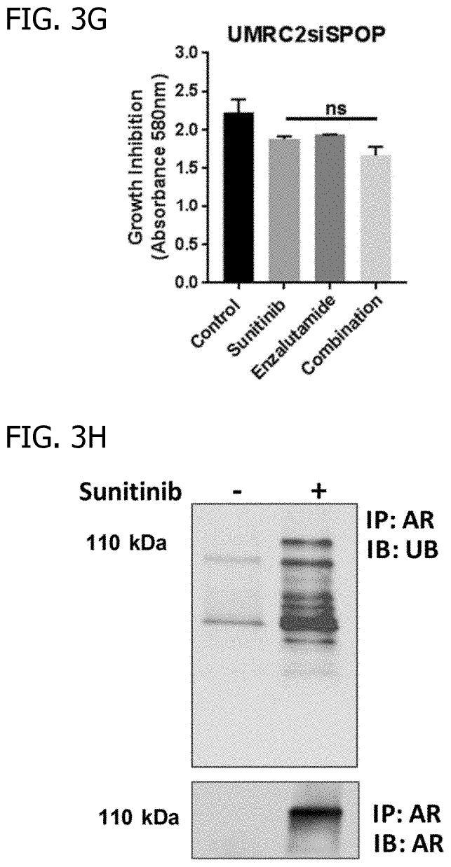

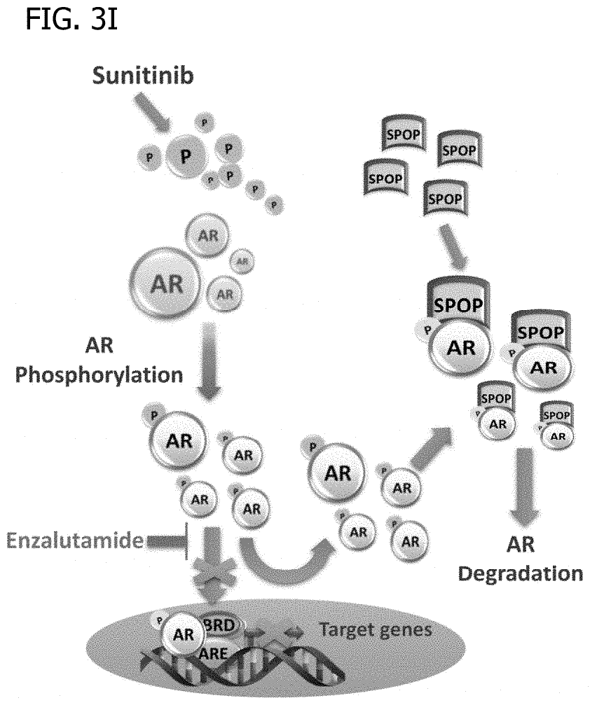

To determine the effect of enzalutamide on AR activity/expression in our system, RCC cells were treated with sunitinib, enzalutamide, or a combination of sunitinib and enzalutamide. AR expression was measured by immunofluorescence. Baseline AR expression was high in 786-0R and UMRC2 cells, and was increased by sunitinib treatment (FIG. 3A). However, concomitant treatment with enzalutamide abrogated sunitinib-induced AR expression. Surprisingly and unexpectedly, a significant decrease in AR expression in cells treated with enzalutamide alone was not observed. To determine whether enzalutamide-induced AR inhibition was due to induced protein degradation, a separate experiment was conducted where 786-0R cells were exposed to enzalutamide, sunitinib, or combination of enzalutamide and sunitinib in the presence or the absence of the proteasome inhibitor MG132. Visual and quantitative data showed no significant decrease in AR expression with enzalutamide or the combination treatment, in the presence of MG132, indicating that enzalutamide induced AR degradation in the presence of sunitinib (FIGS. 3B and 3C). AR rescued degradation by MG132 in the presence of sunitinib and enzalutamide was associated with restoration of cell proliferation, as indicated by Ki67 staining.

Cullin-RING ligases (CRLs) complexes, specifically CRL3 complex, have been identified as bona fide ubiquitination ligases of AR. Thus, Western blot analysis was performed in the RCC lines to determine the level of SPOP expression. A transient SPOP knockdown was then performed in UMRC2 cells, which expressed the highest levels of SPOP compared to the other RCC cell lines. Upon confirmation of successful knockdown, the effect of SPOP siRNA on AR modulation was analyzed. In the presence of sunitinib, there was a significant increased AR expression in UMRC2siSPOP cells as compared to UMRC2, while enzalutamide failed to abrogate this surge in UMRC2siSPOP cells (FIGS. 3D and 3E). The lack of AR degradation induced by enzalutamide in UMRC2siSPOP cells in the presence of sunitinib was associated with loss of the anti-proliferative effect of this combination (FIGS. 3F and 3G). Rescued enzalutamide-induced AR degradation by siSPOP was not associated with restored global serine and tyrosine phosphorylation in the presence of sunitinib, indicating that AR phosphorylation and activation may be part of an epigenetic reprogramming but it does not affect global protein phosphorylation. Overall, these data indicated that SPOP may mediate enzalutamide induced AR degradation, primarily in the presence of sunitinib in RCC. To further investigate the mechanisms responsible for enzalutamide-induced AR degradation, the interaction of AR and ubiquitin was assessed in 786-0R cells treated with sunitinib, following drug wash-out. Immunoprecipitation studies showed that ubiquitin associated with AR following sunitinib treatment (FIG. 3H), indicating the involvement of AR ubiquitination in its proteasome-dependent degradation.

To determine whether the effect of enzalutamide was specifically due to binding to AR, a competitive assay was performed using ddihydrotestosterone (DHT). Following drug wash-out in charcoal stripped media, 786-0R cells were exposed to DHT, and AR nuclear localization was observed. Sunitinib alone also induced strong AR nuclear localization which was abrogated with concomitant enzalutamide treatment. However, concomitant DHT treatment completely rescued the effect of enzalutamide on AR. Taken together, these results indicated that sunitinib induced AR phosphorylation in RCC, and upon AR binding, enzalutamide likely inhibited AR nuclear translocation and/or induced conformational changes that were prone to SPOP-mediated AR degradation via the ubiquitin-proteasome pathway (FIG. 3I).

### Example 4

In this Example, the effect of sunitinib in combination with enzalutamide was determined.

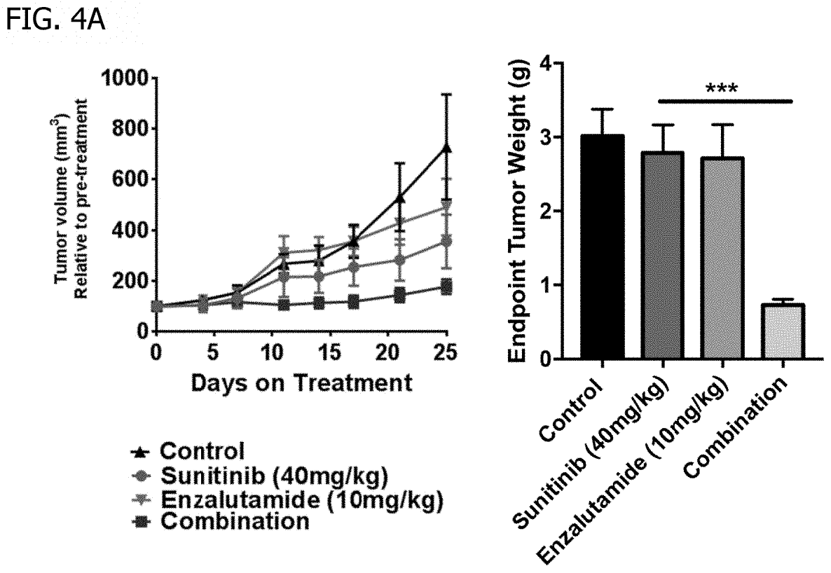

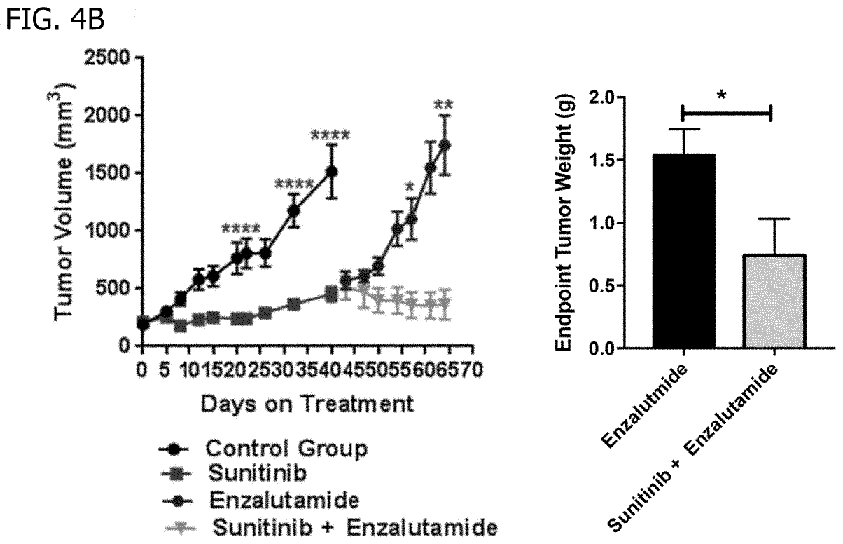

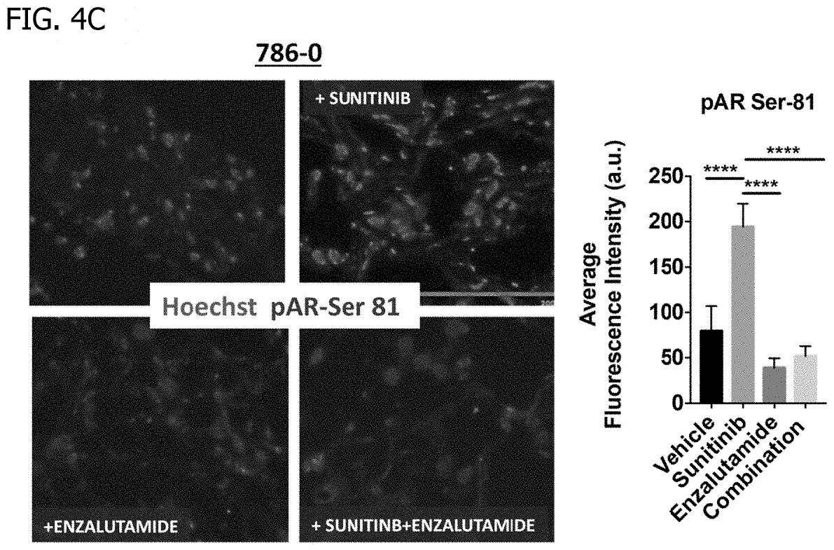

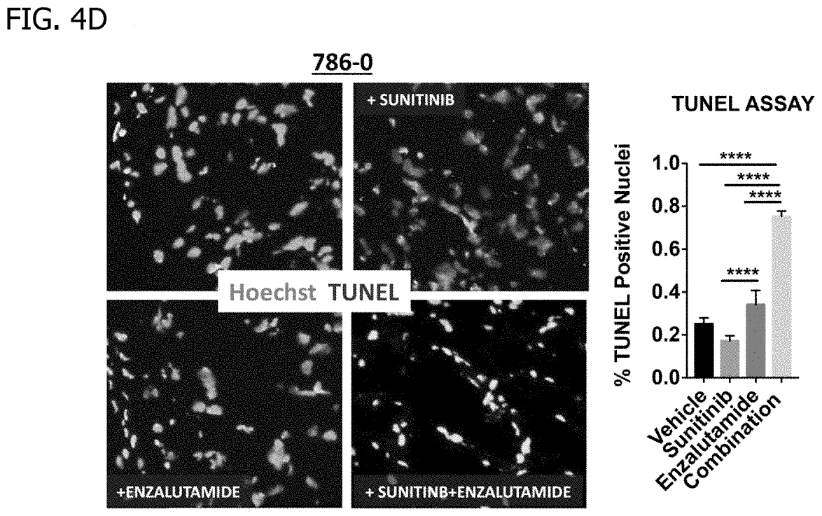

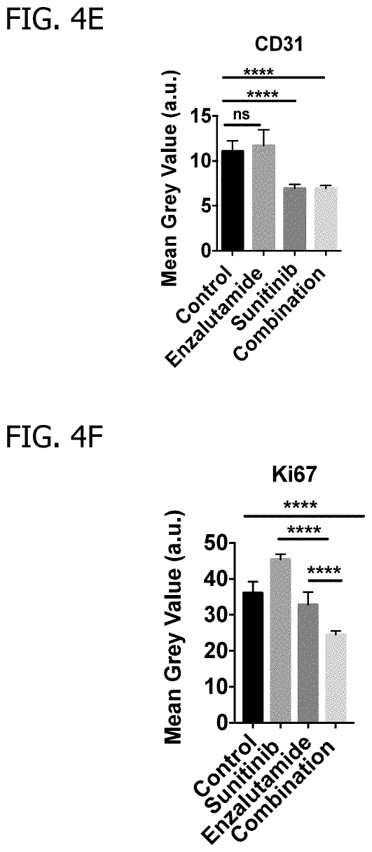

To test the effectiveness of sunitinib in combination with enzalutamide in vivo, independent experiments using the 786-0 (sunitinib sensitive) RCC model were performed. In the first experiment, whether the combination of sunitinib and enzalutamide delayed sunitinib-resistance was determined. 786-0 tumor pieces were subcutaneously implanted into male mice, and, once tumors reached an average size of 100 mm³, treatment with sunitinib, enzalutamide, or a combination of sunitinib and enzalutamide was started. A significant delay in acquired resistance to sunitinib was observed in the combination treatment group, without over toxicities (FIG. 4A). Endpoint tumor weights indicated no significant changes in tumor burden within single agent groups, but a significant decrease in the combination group (p>0.001). To investigate the effect of enzalutamide and sunitinib in combination, after tumors acquired sunitinib resistance, 786-0 (sunitinib sensitive) tumors were implanted, and when tumors were established and reached an average size of 150 mm³, mice were randomly grouped into 2 initial groups, control (n=10) and sunitinib treatment (n=20). Sunitinib treatment was begun and tumor growth was observed until day 45, when tumors became resistant to sunitinib (250% increase volume from nadir) (FIG. 4B). Then, mice in the sunitinib group were further sub-grouped into either sunitinib plus enzalutamide treatment arm (n=10) or enzalutamide treatment arm (n=10).

Tumor growth curves and endpoint tumor weights indicated that tumors in mice treated with sunitinib plus enzalutamide regressed in size, compared to single agent enzalutamide (FIG. 4B). Furthermore, assessment of AR pSer81 expression across treatment groups showed an increase with sunitinib resistance, as compared to the control and the combination treatment group (FIG. 4C). Decreased AR pSer81 expression in the combination group was associated with increased apoptosis (TUNEL) (FIG. 4D). Despite inhibition of angiogenesis (CD31 staining), sunitinib-resistant tumor cells continued to proliferate in vivo (Ki67 staining), though the combination group showed the lowest proliferation rate (FIGS. 4E and 4F).

### Example 5

In this Example, the effect of sunitinib in combination with enzalutamide on circulating kallikrein 2 was determined.

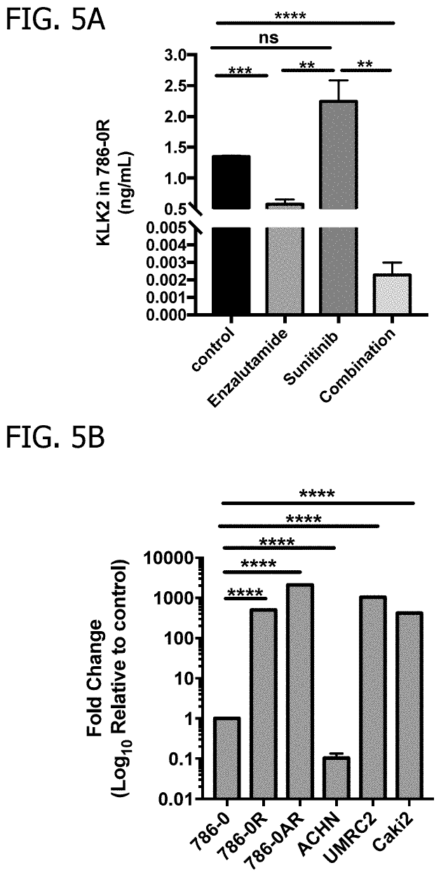

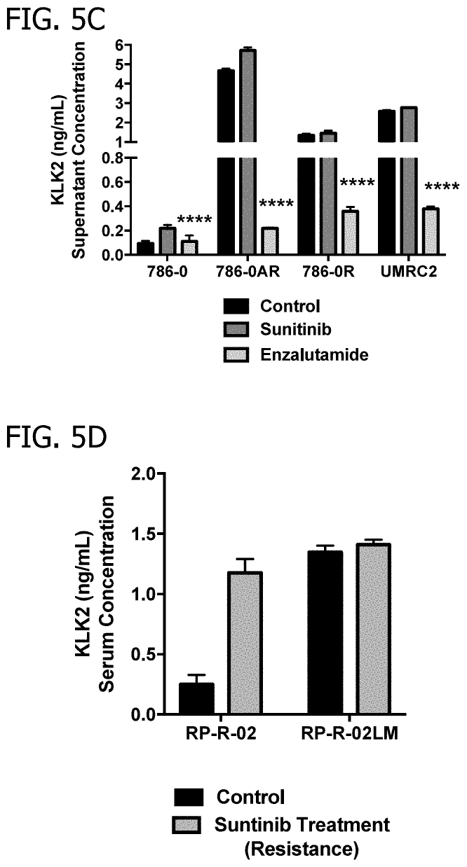

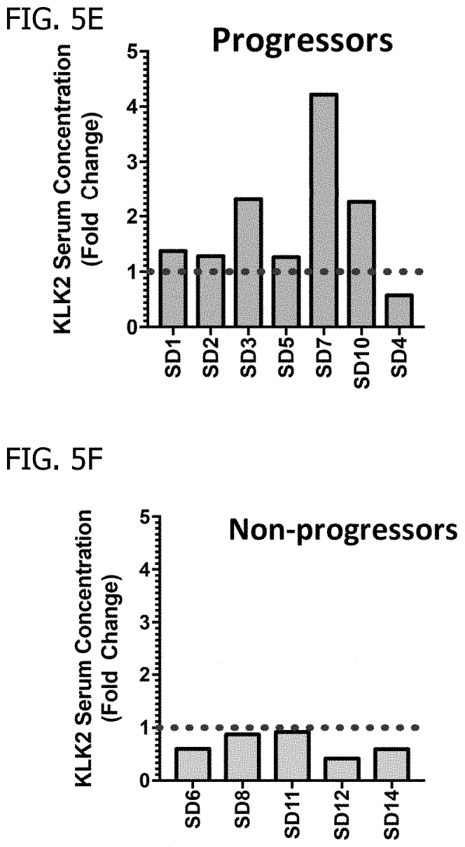

In the original screening of AR target genes, human kallikrein 2 (hK2 or KLK2) and human kallikrein4 (hK4 or KLK4) were increased in 786-R cells. Thus, KLK2 was measured in the models. To determine whether AR activity was effectively inhibited with enzalutamide treatment, KLK2 was measured in conditioned media from the in vitro studies and in serum from the in vivo studies using a human KLK2 ELISA kit. Circulating KLK2 was detected in the 786-0 model, and the levels were decreased in the enzalutamide treated mice, and more significantly in the combination group (FIG. 5A). In tissue culture supernatants, the amount of KLK2 was associated with AR status in RCC cell lines (FIG. 5B), was in vitro modulated by enzalutamide (FIG. 5C), and was increased in the serum in the sunitinib acquired (RP-R-R02) and intrinsic (RP-R-02LM) resistance models (FIG. 5D). Interestingly, in a small number of patients enrolled in our Phase I clinical trial with sunitinib and deltaparin, ccRCC patients who had disease progression, presented increased serum levels KLK2, as compared to non-progressors (defined as patients with either stable disease or objective response as best response) following 3 months treatment (FIGS. 5E and 5F).

### Example 6

In this Example, the effect of sunitinib on AR phosphorylation in prostate cancer was determined.

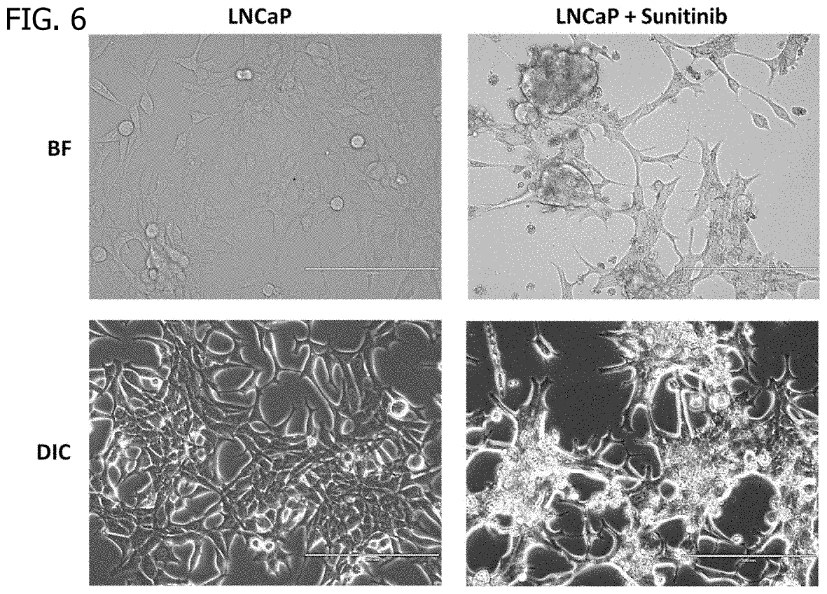

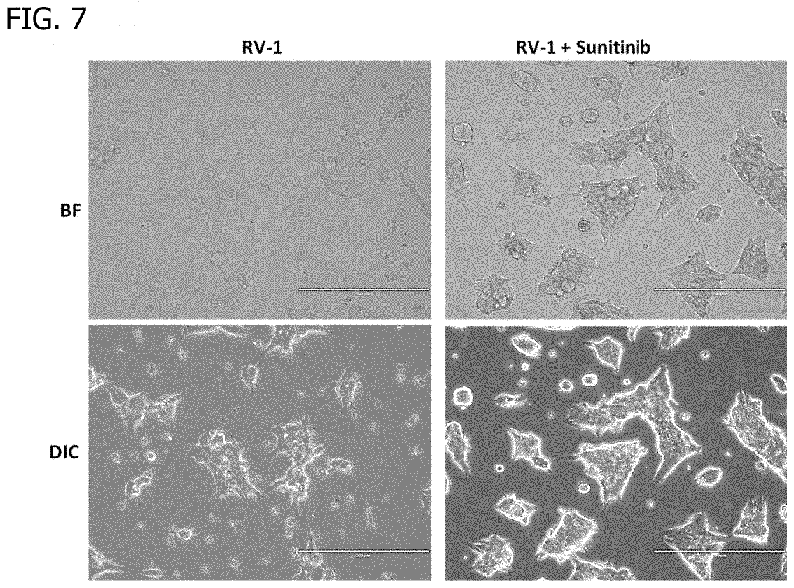

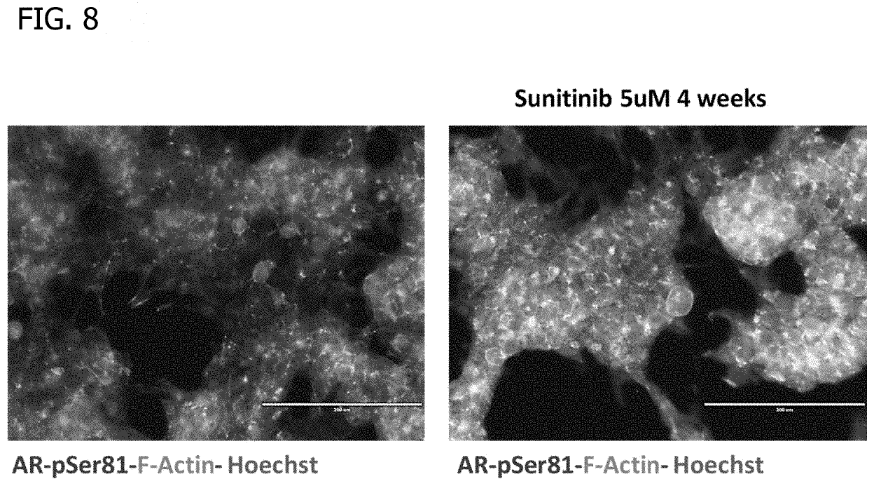

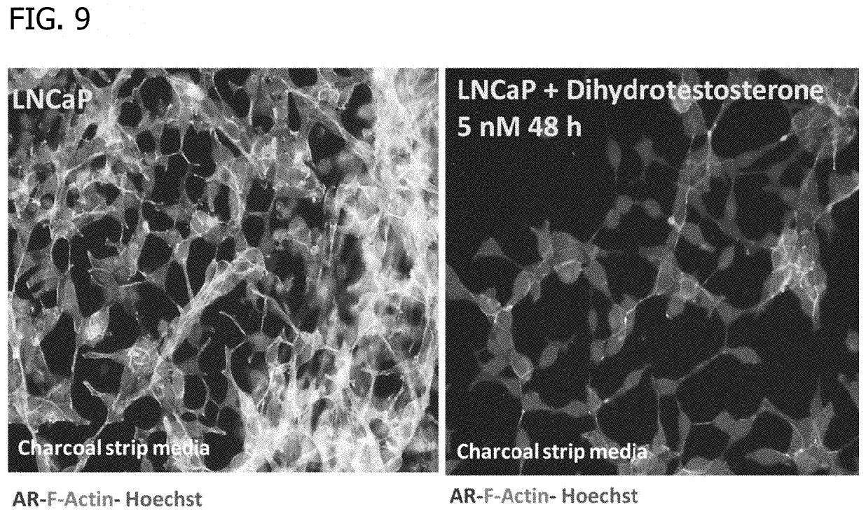

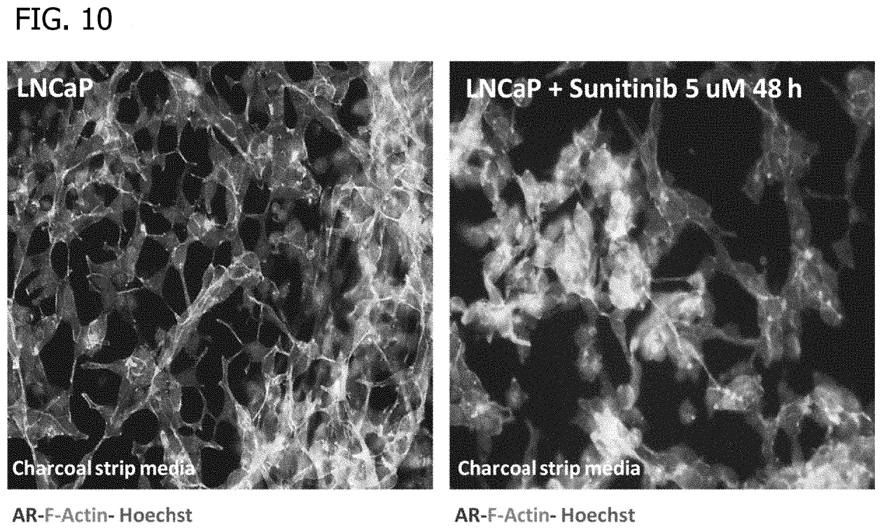

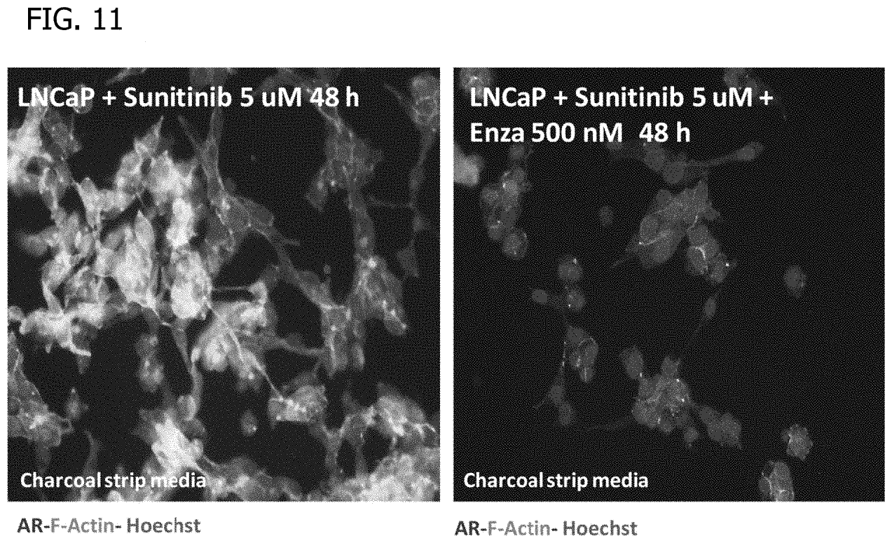

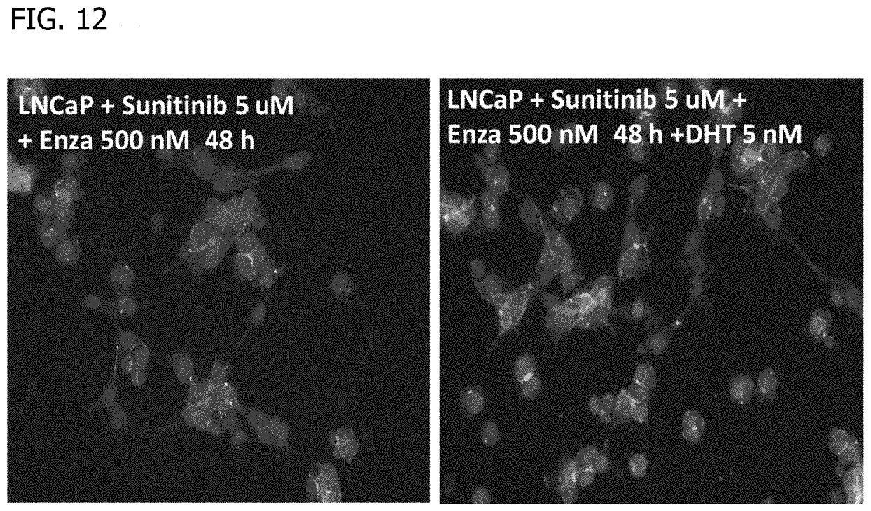

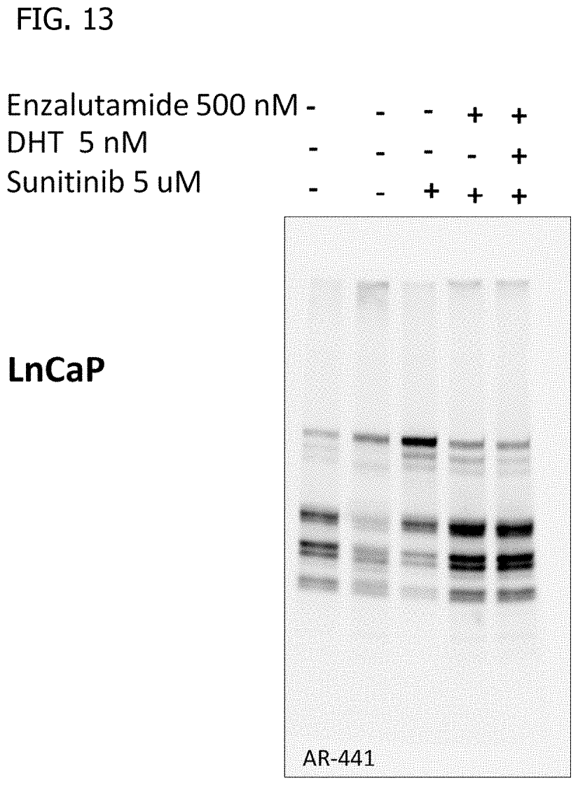

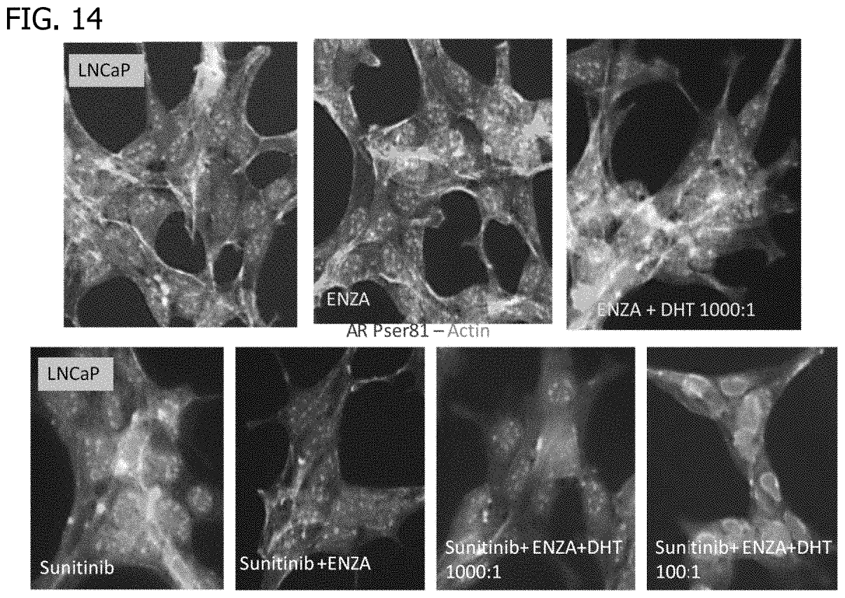

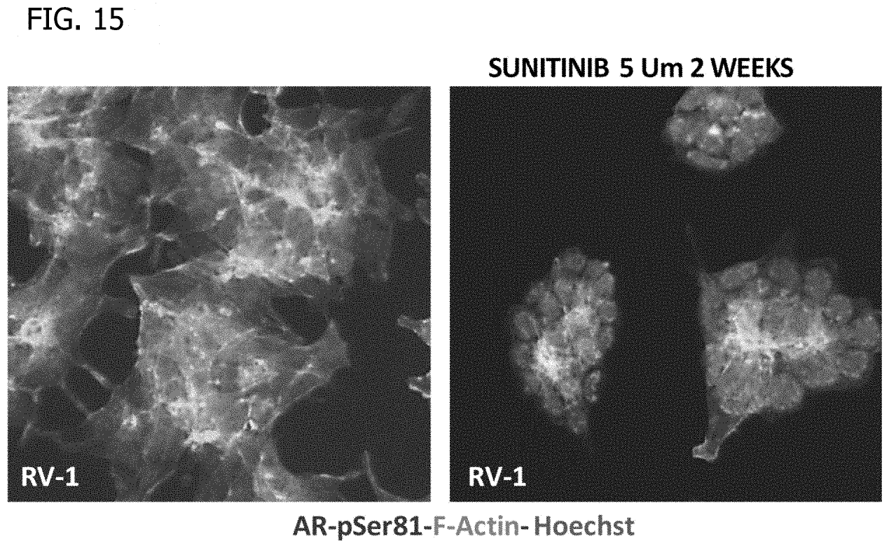

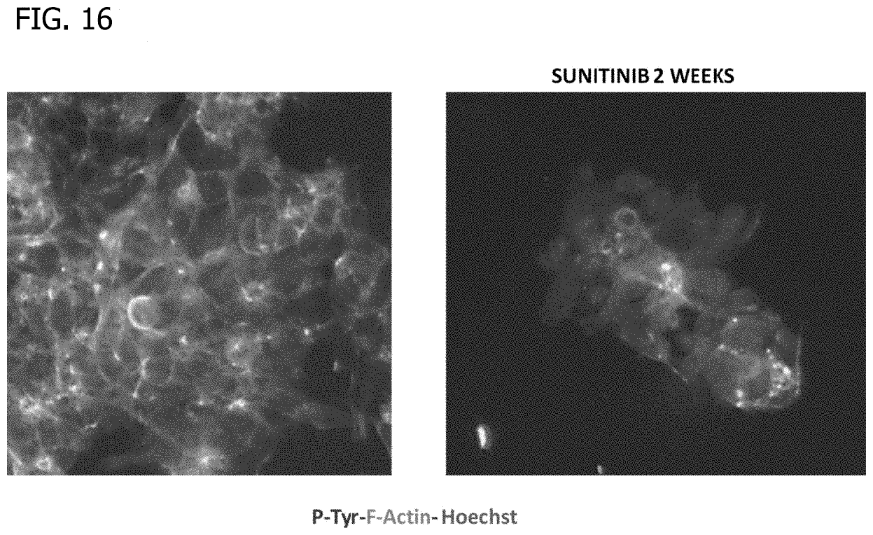

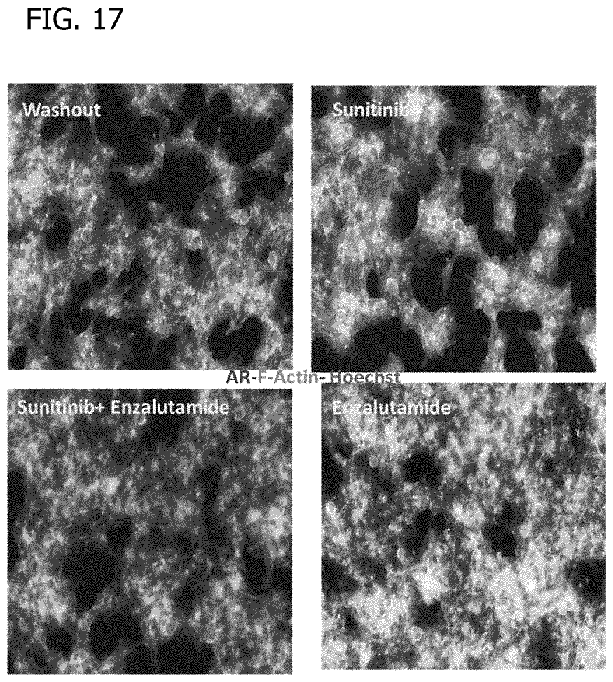

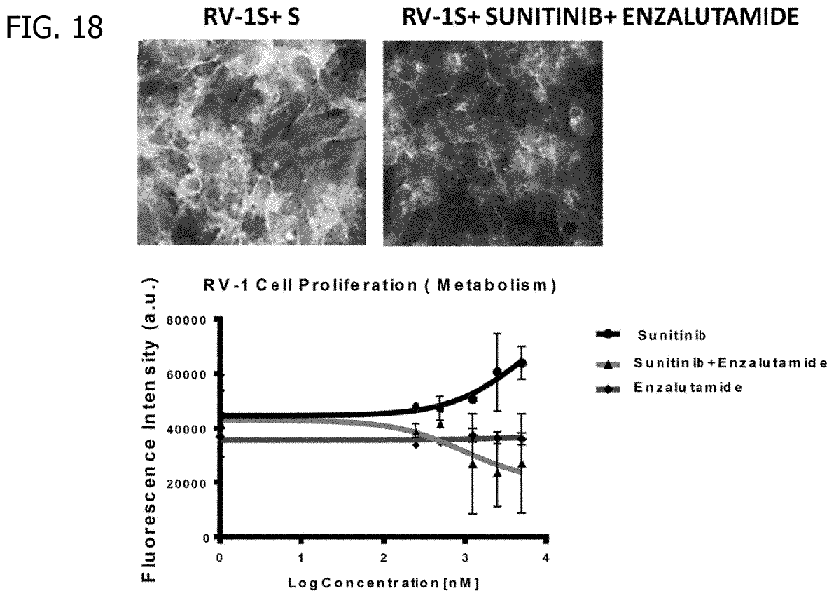

Chronic exposure to sunitinib induced phenotypic changes (cell plasticity) in LnCAP (AR+) cells (FIG. 6) and RV-1 (ARV7+) cells (FIG. 7) as shown by bright field microscopy and increased refractive index. Treatment with sunitinib also induced AR phosphorylation at the Se-81 residue as shown by immunofluorescence (FIG. 8). The localization of full length AR in the nucleus upon LNCaP cells exposure to DHT and was ligand-dependent (FIG. 9). However, exposure to sunitinib induced nuclear localization of AR independently of the presence of the ligand (DHT) (FIG. 10). However, AR nuclear localization induced by sunitinib was decreased by concomitant exposure to enzalutamide (FIG. 11). Enzalutamide also prevented DHT induced AR nuclear localization, as shown in FIG. 12. This observation was confirmed by Western Blot analysis (FIG. 13). Interestingly, sunitinib increased the activity of enzalutamide in sunitinib-exposed LNCaP cells (FIG. 14). When the RV1 cell line which carries the AR spliced variant V7 was examined, chronic exposure to sunitinib increased AR phosphorylation at the Ser-81 residue (FIG. 15). Sunitinib exposure was also associated with increased global tyrosine phosphorylation (FIG. 16). When the cells were treated with enzalutamide in the presence of enzalutamide AR nuclear localization was impaired (FIG. 17). More importantly, in the presence of sunitinib, enzalutamide-resistant RV1 cells reacquired sensitivity to enzalutamide as shown by the decrease in cell proliferation (FIG. 18).

### Example 7

In this Example, plasma KLK-2 measurements in patients with renal cell carcinoma who have received TKIs was determined.

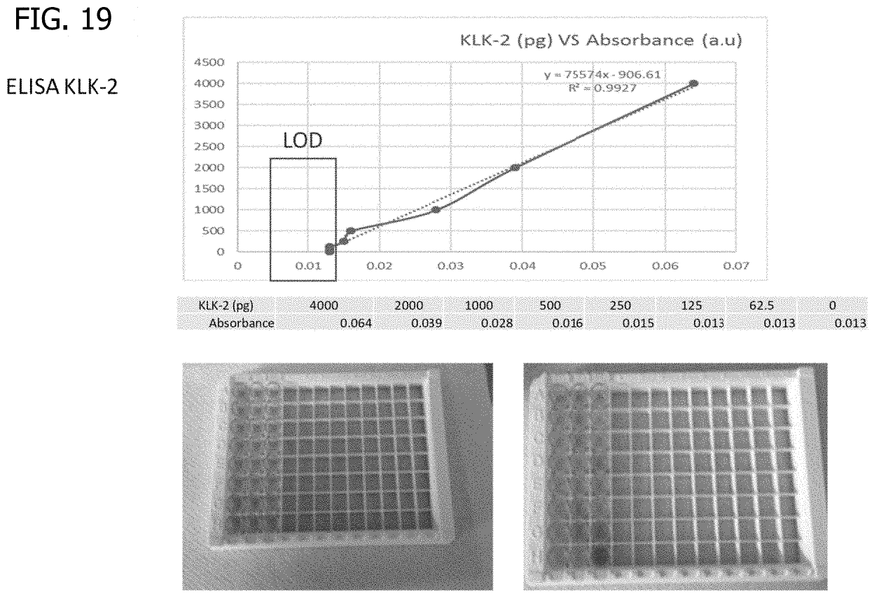

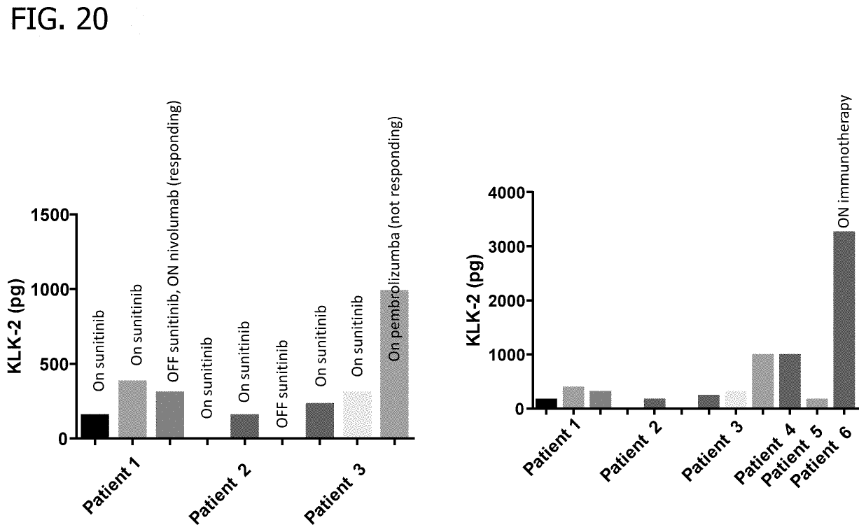

As shown in FIG. 19, a commercially available ELISA kit was used to measure KLK2 in the plasma of kidney cancer patients treated with sunitinib. In patients treated with sunitinib, an increase of KLK2 was observed, which indicates AR activation secondary to drug resistance (FIG. 20). Interestingly, elevated levels were also observed in patients who were treated with immune-checkpoint inhibitors and progressing, indicating that KLK2 may be also a biomarker for tumor burden.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A composition comprising an androgen receptor antagonist and a kinase inhibitor.

2. The composition of claim 1, wherein the androgen receptor antagonist is chosen from enzalutamide, bicalutamide, apalutamide, abiraterone and ODM-201.

3. The composition of claim 1, wherein the kinase inhibitor is chosen from a receptor tyrosine kinase inhibitor, a serine/threonine kinase inhibitor, a lipid kinase inhibitor, and combinations thereof.

4. The composition of claim 3, wherein the receptor tyrosine kinase inhibitor is chosen from sunitinib, axitinib, pazopanib, cabozantinib, lenvatinib, sorafenib, regorafenib, imatinib, dasatinib, nilotinib, bosutinib, ponatinib, ruxolitinib, tofacitinib, gefitinib, erlotinib, lapatinib, vandetanib, afatinib, nintedanib, crizotinib, ceritinib, and ibrutinib.

5. The composition of claim 3, wherein the serine/threonine kinase inhibitor is chosen from vemurafenib, trametinib, sirolimus, temsirolimus, everolimus, and palbociclib.

6. The composition of claim 3, wherein the lipid kinase inhibitor is chosen from idelalisib and buparlisib.

7. A method of treating a subject having or suspected of having renal cell carcinoma, the method comprising: administering to the subject a composition comprising an androgen receptor antagonist.

8. The method of claim 7, wherein the subject has or is suspected of having drug resistant renal cell carcinoma.

9. The method of claim 7, wherein the subject is receiving or has received a receptor tyrosine kinase inhibitor therapy.

10. The method of claim 7, wherein the androgen receptor antagonist is chosen from enzalutamide, bicalutamide, apalutamide, abiraterone and ODM-201.

11. (canceled)

12. (canceled)

13. (canceled)

14. (canceled)

15. (canceled)

16. (canceled)

17. (canceled)

18. (canceled)

19. (canceled)

20. (canceled)

21. A method of identifying a subject having or suspected of having drug resistant renal cell carcinoma, the method comprising: obtaining a sample from the subject; and detecting androgen receptor expression, detecting androgen receptor phosphorylation, and combinations thereof.

22. (canceled)

23. (canceled)

24. A method of treating a subject having or suspected of having cancer, the method comprising: administering to the subject a composition comprising an androgen receptor antagonist.

25. (canceled)

26. (canceled)

27. (canceled)

28. (canceled)

29. (canceled)

30. (canceled)

31. (canceled)

32. (canceled)

33. (canceled)

34. (canceled)

35. A method of identifying a subject having or suspected of having drug resistant prostate cancer, the method comprising: obtaining a sample from the subject; and detecting androgen receptor expression, detecting androgen receptor phosphorylation, and combinations thereof.

36. (canceled)

37. A method of treating a subject having or suspected of having a solid tumor, the method comprising: administering to the subject a composition comprising an androgen receptor antagonist and a kinase inhibitor.

38. (canceled)

39. (canceled)

40. (canceled)

41. (canceled)

42. (canceled)

43. (canceled)

44. (canceled)

45. Use of a composition comprising an androgen receptor antagonist and a kinase inhibitor to treat cancer.

46. (canceled)

47. (canceled)

48. (canceled)

49. (canceled)

50. (canceled)

51. (canceled)

52. (canceled)

53. (canceled)


SEQUENCE LISTING (2 sequences)

SEQ ID NO: 1 - DNA, 20 bp, Artificial Sequence
  Synthetic
ggtgagcagagtgccctatc

SEQ ID NO: 2 - DNA, 20 bp, Artificial Sequence
  Synthetic
tcgggtatttcgcatgtccc